(19) United States
(12) Patent Application Publication
(10) Pub. No.: US 2015/0343021 A1
(43) Pub. Date: Dec. 3, 2015

(54) METHODS FOR TREATING CANCER USING COMPOSITIONS COMPRISING HSS1 AND/OR HSM1

(71) Applicant: Neumedicines, Inc., Pasadena, CA (US)

(72) Inventors: Lena A. Basile, Tujunga, CA (US); Christopher Edward Lawrence, San Francisco, CA (US)

(73) Assignee: Neumedicines, Inc., Pasadena, CA (US)

(21) Appl. No.: 14/726,247

(22) Filed: May 29, 2015

Related U.S. Application Data
(60) Provisional application No. 62/004,554, filed on May 29, 2014.

(51) Int. Cl. A61K 38/17; A61K 45/06; A61N 5/10; A61K 41/00

(52) U.S. Cl. CPC A61K 38/1709; A61K 41/0038; A61K 45/06; A61N 5/10

(57) ABSTRACT

The present application relates to methods of treating cancer using compositions comprising Hematopoietic Signal peptide-containing Secreted 1 (HSS1), derivatives of HSS1. Hematopoietic Signal peptide-containing Membrane domain-containing 1 (HSM1), derivatives of HSM1, or any combination thereof.

### CROSS-REFERENCE TO RELATED APPLICATION

This application claims priority to U.S. Provisional Patent Application No. 62/004,554, filed on May 29, 2014, the disclosure of which is specifically incorporated by reference in its entirety.

### FIELD OF THE INVENTION

The present invention is directed to methods for treating cancer using compositions comprising Hematopoietic Signal peptide-containing Secreted 1 (HSS1) and/or Hematopoietic Signal peptide-containing Membrane domain-containing 1 (HSM1).

### BACKGROUND

Although little is known about Human Hematopoietic Signal peptide-containing Secreted 1 (hHSS1), there is evidence that hHSS1 is one of the glucose-responsive genes with both mRNA and protein secretion being regulated by glucose. Wang et al., “Molecular cloning of a novel secreted peptide, INM02, and regulation of its expression by glucose,” J. Endocrinol., 202(3):355-364 (2009). As such, it is speculated that hHSS1 could be associated with the functions of pancreatic islets, specifically beta-cells. Id. Recently, hHSS1 was identified as endoplasmic reticulum (ER) membrane protein complex subunit 10 (EMC10), one of the components of ER associated degradation (ERAD), an ubiquitin and proteasome dependent process. Christianson et al., “Defining human ERAD networks through an integrative mapping strategy,” Nat. Cell. Biol., 14(1):93-105 (2011). The mouse orthologue of hHSS1 (C19orf63) is the only gene that is highly expressed in mice with the 22q11.2 microdeletion, an animal model used to study the association between 22q11.2 microdeletion and a strong risk for schizophrenia development. Xu et al., “Derepression of a neuronal inhibitor due to miRNA dysregulation in a schizophreniarelated microdeletion,” Cell, 152(1-2):262-275 (2013). Up-regulation of Mirta 22, the mouse orthologue of hHSS1, was shown to be responsible for abnormal neuronal morphology through the inhibition of neuronal connectivity, again linked to schizophrenia susceptibility and cognitive deficit. Id. It was also verified that Mirta 22 expression was purely neuronal and located in the Golgi apparatus. Id.

It was previously demonstrated that ectopic overexpression of hHSS1 has a negative modulatory effect on cell proliferation and tumorigenesis, in both in vitro and in vivo murine model of glioblastoma. Junes-Gill et al., “hHSS1: a novel secreted factor and suppressor of glioma growth located at chromosome 19q13.33,” J. Neurooncol., 102(2):197-211 (2011). However, the molecular mechanism by which hHSS1 suppresses cell proliferation and tumorigenesis has yet to be defined.

The National Cancer Institute (NCI) estimates that 22,340 new cases and 13.110 deaths from brain and other nervous system cancers occurred in US in 2011. Malignant gliomas are the most common and most aggressive primary brain tumor, accounting for more than half of the new cases of primary malignant brain tumors diagnosed each year in US. Moore K, Kim L., “Primary Brain Tumors: Characteristics, Practical Diagnostic and Treatment Approaches,” in Glioblastoma: Molecular Mechanisms of Pathogenesis and Current Therapeutic Strategies, Edited by Ray SK pp 43-75 (2010). Given the fatal effect of most neurological and brain cancers, novel approaches are needed to increase survival rate of patients diagnosed with these diseases.

Methods of treating brain cancer using HSS (Hematopoietic Signal peptide-containing Secreted 1), HSM1 (Hematopoietic Signal peptide-containing Membrane domain-containing 1), or a combination thereof have previously been described. See U.S. Pat. No. 8,735,342.

Contemporary treatment modalities do not substantially increase the survival rate and generally are not curative. There is a critical need to elucidate novel pathways and factors involved in the inhibition of tumor growth in glioma to facilitate the development of novel anti-tumoral therapeutics that may be key in controlling and, eradicating malignant glioma. Identifying and characterizing novel proteins, such as hHSS1, opens up the possibility of discovering such novel biological functions and pathways. Thus, it is critical to characterize and dissect the anti-tumoral effect of hHSS1.

### SUMMARY OF INVENTION

The present invention is directed to methods of treating cancer comprising administering a therapeutically effective amount of Hematopoietic Signal peptide-containing Secreted 1 (HSS1 or human hHSS1), Hematopoietic Signal peptide-containing Membrane domain-containing 1 (HSM1 or human hHSM1), or a combination thereof, as either a solo therapy or as an adjuvant to a conventional cancer therapy used now or later discovered. Embodiments of the invention described herein can be combined.

Included in the definition of “conventional cancer therapies” are all forms of radiation therapy and all forms of chemotherapies, which can be used in conjunction with various forms of radiation therapy. These conventional cancer therapies also include surgery to remove all or part of the cancerous tissue, along with any combination of radiation and chemotherapy.

When used as an adjuvant to chemotherapy, HSS1 and/or HSM1 can be administered before, during, or after chemotherapy, or any combination of before, during, or after chemotherapy. When used as an adjuvant to radiation therapy, HSS1 and/or HSM1 can be administered before, during, or after chemotherapy, or any combination of before, during, or after radiation therapy.

The dose of HSS1 and/or HSM1 used in the present invention is the dose required to be efficacious as well as safe, regardless of how HSS1 and/or HSM1 is delivered.

In yet another embodiment of the invention. HSS1 and/or HSM1 can inhibit tumor angiogenesis.

In yet another embodiment of the invention, HSS1 and/or HSM1 can radiosensitize a tumor or cancer, leading to more effective radiation treatment of the tumor or cancer.

In another embodiment, the patient population to be treated is a carrier of BRCA1-2.

The methods and compositions of the invention can increase the survival of patients diagnosed with cancer. Other benefits include a reduction in tumor mass, more complete cancer remission, and/or slowing or inhibition of tumor growth, progression, and/or metastasis.

In one embodiment of the invention, the cancer to be treated is a brain cancer. In this embodiment, HSS1 and/or HSM1 are used as an adjuvant to a conventional cancer therapy. Exemplary active agents used to treat gliomas or brain cancers include, but are not limited to, endostatin and angiostatin.

The brain cancer can be a primary or secondary brain cancer (a secondary brain cancer is a brain cancer which has metastatized from a non-brain cancer). The preferred brain cancer treatable with embodiments of the present invention is glioma, particularly glioblastoma multiforme. Other brain cancers are also treatable with the present invention, including but not limited to astrocytoma, oligodendroglioma, ependymoma, meningiomas, neuroblastoma, acoustic neuroma/schwannomas, and medulloblastoma.

In one embodiment of the invention, the brain cancer to be treated with a method of the invention is a secondary brain cancer which has metastatized from a non-brain cancer.

In another embodiment of the invention, HSS1 and/or HSM1 are used as a solo antiangiogenic cancer therapy, or as an adjuvant to a conventional cancer therapy, wherein the cancer is not a brain cancer. The HSS1 and/or HSM1 can target endothelial cell neovascularization, resulting in slowing or inhibiting tumor growth, progression, and/or metastasis.

Any non-brain cancer can be treated using the compositions and methods of the invention in either a solo or combination therapy. In one embodiment, the non-brain cancer is ovarian cancer, pancreatic cancer, or breast cancer.

In another embodiment of the invention encompassed are methods for treating inflammatory diseases by reducing inflammatory cell invasion by anti-angiogenic activity, comprising administering to a subject in need a composition according to ther invention. The composition can comprise, for example, at least one compound selected from the group consisting of (a) Hematopoietic Signal peptide-containing Secreted 1 (HSS1); (b) Hematopoietic Signal peptide-containing Membrane domain-containing 1 (HSM1); (c) a peptide having at least about 80% homology to HSS1, wherein the peptide exhibits anti-angiogenic activity; (d) a peptide having at least about 80% homology to HSM1, wherein the peptide exhibits anti-angiogenic activity; (e) a HSS1 fragment comprising at least 4 contiguous amino acids from the HSS1 amino acid sequence, wherein the fragment exhibits anti-angiogenic activity; (f) a HSM1 fragment comprising at least 4 contiguous amino acids from the HSM1 amino acid sequence, wherein the fragment exhibits anti-angiogenic activity; or (g) any combination thereof. The inflammatory disease to be treated can be any known inflammatory disease, including but not limited to Arthritis, Crohn's disease, Psoriasis, or Endometriosis.

Any pharmaceutically acceptable delivery method now known or developed in the future can be utilized in the methods of the invention to deliver HSS1 and/or HSM1 to the cancer patient, either locally or systemically. Further, if a brain cancer is to be treated, any methods now known or developed in the future that facilitate passage across the blood brain barrier can be utilized in the methods of the invention to deliver HSS1 and/or HSM1 to the site of the brain cancer (although local delivery to the brain cancer is not required). Other delivery methods included in the present invention are delivery via liposomes and fusion proteins. When a brain cancer is to be treated, HSS1 and/or HSM1 can be formulated as a pharmaceutical for systemic delivery or for delivery to the brain by intracerebroventricular infusion, or any other like delivery method.

Another form of delivery method for the HSS1 and/or HSM1 is via various gene therapy vector delivery systems available in the art or to be discovered in the future. In one embodiment of the invention, the gene therapy vector is derived from adenovirus. In another embodiment of the invention, the gene therapy vector is derived from the herpes virus. In still another embodiment of the invention, the gene therapy vector is derived from a retrovirus.

Also encompassed are pharmaceutical compositions useful in the methods of the invention. The compositions comprise HSS1, HSM1, or a combination thereof. The compositions comprise (a) Hematopoietic Signal peptide-containing Secreted 1 (HSS1); (b) Hematopoietic Signal peptide-containing Membrane domain-containing 1 (HSM1); (c) a peptide having at least about 80% homology to HSS1, wherein the peptide exhibits anti-angiogenic activity; (d) a peptide having at least about 80% homology to HSM1, wherein the peptide exhibits anti-angiogenic activity; (e) a HSS1 fragment comprising at least 4 contiguous amino acids from the HSS1 amino acid sequence, wherein the fragment exhibits anti-angiogenic activity; (f) a HSM1 fragment comprising at least 4 contiguous amino acids from the HSM1 amino acid sequence, wherein the fragment exhibits anti-angiogenic activity; or (g) any combination thereof. The compositions can additionally comprise at least one pharmaceutically acceptable carrier. In another embodiment, the pharmaceutical compositions can additionally comprise at least one active agent which is not HSS1 or HSM1, wherein the active agent is useful in treating a cancer.

In another embodiment of the invention, encompassed are pharmaceutical compositions useful in the cancer treatment methods of the invention. The compositions comprise a peptide having at least about 80% homology to HSS1, a peptide having at least about 80% homology to HSM1, or any combination thereof. In addition, the invention encompasses compositions comprising at least one HSS1 fragment, HSM1 fragment, or a combination of at least one HSS1 fragment and at least one HSM1 fragment. Thus, the invention encompasses pharmaceutical compositions comprising a therapeutically effective amount of HSS1, HSM1, at least one HSS1 fragment, at least one HSM1 fragment, a peptide having at least about 80% homology to HSS1 (or a homology as defined herein), a peptide having at least about 80% homology to HSM1 (or a homology as defined herein), or any combination thereof.

The foregoing general description and following detailed description are exemplary and explanatory and are intended to provide further explanation of the invention as claimed. Other objects, advantages, and novel features will be readily apparent to those skilled in the art from the following detailed description of the invention.

### DESCRIPTION OF THE FIGURES

FIGS. 1A. 1B, and 1C show molecular networks of genes up- and down-regulated in U87 and A172 cells overexpressing hHSS1 in comparison with mock-stable transfected cells. Network of genes based on connectivity identified by IPA analysis. FIG. 1A: Top gene network of U87 cells depicting genes involved in cell cycle, cell death, DNA replication, recombination and repair. ANKRD1 was the most up-regulated gene. Many genes with direct and indirect relationship with E2F gene were down-regulated by HSS1. FIG. 1B: Top gene network of A172-hHSS1 clone #7 showing genes involved in cell cycle, cellular assembly and organization, DNA replication, recombination and repair. Several genes down-regulated by hHSS1 in A172 clone #7 are target genes regulated by VEGF. FIG. 1C: Top gene network of A172-hHSS1 clone #8 showing genes involved in tissue morphology and cellular development. Some of the hHSS1 modulated genes in A172 clone #8 are responsible for ERK regulation. Different shapes of the nodes (genes/gene products) represent the functional classes of the gene products and the lines represent the biological relationships between the nodes. The length of an edge reflects the evidence in the literature supporting that node-to-node relationship. The intensity of the node color indicates the degree of up- (red) or down-regulation (green) of the respective gene. Gray represents a gene related to the others that did not meet the cutoff criteria. A solid line without arrow indicates protein-protein interaction. Arrows indicate the direction of action (either with or without binding) of one gene to another.

FIG. 2 shows that the role of BRCA in the DNA damage response pathway is regulated by hHSS1-overexpression in U87 cells by iReport analysis. Blue color indicates down-regulation of a gene, orange color indicates up-regulation of a gene.

FIGS. 3A, 3B, 3C, and 3D show that the mitotic roles of polo-like kinase pathway is regulated by hHSS1-overexpression in U87 cells by iReport analysis. Blue color indicates down-regulation of a gene, orange color indicates up-regulation of a gene. FIG. 3A shows centrosome separation and maturation; FIG. 3B shows mitotic entry; FIG. 3C shows septum-inducing network and cytokinesis; and FIG. 3D shows metaphase to anaphase transition and mitotic exit.

FIGS. 4A, 4B, and 4C show validation of selected genes differentially expressed by hHSS1 overexpression. Dark color indicates genes validated by qRT-PCR. Light color indicates genes differentially expressed by microarray analysis. FIG. 4A=Genes differentially expressed in U87 cells. FIG. 4B=Genes differentially expressed in A172-hHSS1 C#7 and FIG. 4C=A172-hHSS1 C#8.

FIGS. 5A and 5B show the effect of hHSS1 on cell cycle phases for glioma cells. Cell cycle analysis was performed by propidium iodide staining followed by flow cytometry using day 4 and 5 from a U87 and A172 growth curve, respectively. Columns represents mean percentage of cells in each phase of the cell cycle ±SEM (n=2), p<0.05, one way ANOVA with post hoc pairwise Tukey test. A) Cell cycle analysis in U87 cells (FIG. 5A) and A172 cells (FIG. 5B).

FIGS. 6A and 6B show that overexpression of hHSS1 significantly decreases the migration and invasion of U87 cells, and the migration of A172 cells. FIG. 6A: Transwell migration assay for U87 and A172 cells overexpressing hHSS1 or control vector. FIG. 6B: Matrigel invasion assay for U87 and A172 cells overexpressing hHSS1 or control. 10% FBS serum was added as chemoattractant. After 24 h incubation, cells that migrated through the membrane or invaded through the matrix were fixed, stained with H&E and pictures (200×, magnification) of 9 fields of each replicate was taken for cells counting. Two independent experiments using duplicates were done for each assay. Data shown are mean±SEM. ** P<0.01; *** P<0.001, t-test.

FIGS. 7A and 7B show that overexpression of hHSS1 impacts U87 and A172 tumor-induced HUVEC migration and invasion. FIG. 7A: Transwell migration and invasion assay for HUVEC co-cultured with U87 overexpressing hHSS1 or control vector. FIG. 7B: Transwell migration and invasion assay for HUVEC co-cultured with A172 overexpressing hHSS1 or control vector. Glioma cells were seeded in the bottom chamber containing media with 2% FBS. After 24 h, media was changed to serum-free media supplemented with 0.1% BSA. HUVEC cells were seeded in the upper chamber containing media with 0.1% BSA. A172 or U87 cells were seeded at 10:1 ratio of HUVEC cells. After 24 h, cells that migrated and invaded the matrix were fixed, stained with H&E and pictures (200×, magnification) of 21 fields of each replicate were taken for cells counting. Two independent experiments using duplicates were done for each assay. Data shown are mean±SEM. *** P<0.001, t-test. Black arrow shows net-like formation of invaded cells.

FIGS. 8A, 8B, 8C, and 8D show that purified hHSS1 inhibits HUVEC tube formation in a concentration-related manner. HUVECs growing on top of matrigel were treated with different concentrations of purified hHSS1 or vehicle control (PBS). Cells were pre-treated with hHSS1 protein or vehicle control for 3 h before plating on top of matrigel. After 8 h. cells were stained with crystal violet and tube formation was evaluated. Images (100×, magnification) are representative of two independent experiments done in duplicate. FIGS. 8A and 8C show the inhibitory effect of purified hHSS1 on tube formation using 500 nM (FIG. 8A) and 200 nM (FIG. 8C) of hHSS1 protein. FIGS. 8B and 8D show vehicle control diluted following the protein dilution scheme.

FIGS. 9A and 9B show hHSS1 expression analysis in GBM from the TCGA dataset. FIG. 9A: Correlation analysis between hHSS1 and BRCA2 expression (r=−0.224. P<0.0005). FIG. 9B: Log 2-transformed gene expression levels for selected genes between high and low-hHSS1 expression cohorts. Mean gene expression levels between cohorts were compared by two-tailed Student's t-test, P<0.01. P values −HSS1^{lo} vs. HSS1^{hi}: (hHSS1, P<6.55e⁻⁹⁸), (ADAMTS1, P<0.014), (BRCA2, P<0.00006), Endostatin (COL18A1), P<0.048).

### DETAILED DESCRIPTION OF THE INVENTION

### I. Overview

### Cancer Therapy

The ideal cancer-therapy should be directed at two distinct cell populations, a tumor cell population and an endothelial cell population, each of which can stimulate growth of the other. Folkman J., “Tumor angiogenesis and tissue factor,” Nature Med., 2:167-168 (1996); and O'Reilly et al., “Endostatin: an endogenous inhibitor of angiogenesis and tumor growth,” Cell, 88(2):277-285 (1997). Combined treatment of each cell population may be better than treatment of either compartment alone. Teicher et al., “Potentiation of cytotoxic cancer therapies by TNP-470 alone and with other anti-angiogenic agents,” Int. J Cancer., 57(6):920-925 (1994).

The microarray and in vitro data described herein suggest that hHSS1 protein is involved in the negative regulation of fundamental biological processes such as cell proliferation, migration, invasion, tumorigenesis and angiogenesis. Therefore, hHSS1 can be used as a therapeutic to target not only glioma tumor cells growth, but also endothelial cell neo-vascularization, and can provide a novel therapeutic intervention along with chemotherapy. Thus, The present invention has many uses in the treatment of various cancers.

### II. Microarray and In Vitro Data

The present invention describes global expression profile of A172 and U87 human glioma-derived cells overexpressing hHSS1 to gain insights into the mechanism by which hHSS1 acts on glioma cells and to further elucidate its function. See e.g., Junes-Gill et al., Human hematopoietic signal peptide-containing secreted 1 (hHSS1) modulates genes and pathways in glioma: implications for the regulation of tumorigenicity and angiogenesis,” BMC Cancer, 14:920 (December 2014), which is specifically incorporated by reference. As described in the examples below, microarray analysis was used to determine cellular transcriptional changes in response to 96-120 hours of hHSS1 overexpression in stably transfected cells. Junes-Gill et al., J. Neurooncol., 102(2):197-211 (2011). Focused analysis of these time points allows the identification of early hHSS1 regulated genes involved in the cytostatic effect exerted by hHSS1 in A172 and U87 human glioma-derived cells. Moreover, cDNA microarray analysis can be useful for the elucidation of the key factors in tumorigenesis, and facilitate identification of genes involved in pathways related to hHSS1. This can lead to significant progress in the treatment of human disease by defining new therapeutics and novel molecular targets, particularly in glioma. Analysis of the TCGA database and the effect of hHSS1 on cell cycle, migration and invasion of glioma-derived cells, as well as the effect of hHSS1 on the angiogenic properties of HUVEC are described.

In the study described in the examples, advanced bioinformatics were combined with functional assays to subsequently identify key biological pathways directly or indirectly affected by hHSS1. The observed effect of hHSS1 included DEGs having either stimulatory or inhibitory effects, but ultimately leading to inhibition of tumoral and angiogenic properties. hHSS1 overexpression strongly affected a number of transcriptional regulators, enzymes, growth factors, transporters and extracellular matrix proteins, hence altering important signaling pathways, and impacting biological functions. The pathway analysis approach using IPA and Ingenuity® iReport indicated that hHSS1 plays a role in several biological functions considered hallmarks of cancer, including cell proliferation, cell cycle regulation, DNA replication, DNA repair, angiogenesis, cell migration, and cell invasion.

Previously, it was shown that hHSS1 overexpression negatively regulated proliferation of U87 and A172 cells (Junes-Gill, 2011). The microarray data described herein of the same set of cells evaluated by pathway analysis yielded a similar effect of down regulation of genes involved in proliferation, cell cycle progression and cell division process. Furthermore, the cell cycle analysis demonstrated that the inhibition of U87 cell proliferation was accompanied by a decrease of cells in G0/G1 and a concomitant increase of cells in S and G2/M. The down regulation indicated by microarray analysis of cyclin E, cyclin B, CDC2 and a complex of proteins (BRCA1, BRCA2, Rad51, BARD and FANCD2) responsible for regulating the S and G2 cell cycle phases, might partly explain the inhibitory effect of hHSS1 overexpression on proliferation previously reported for U87 cells.

The IPA top molecular network included ANKRD1 as the most up-regulated gene in U87 cells, a nuclear factor that has negative transcriptional activity in endothelial cells. (Zou et al., 1997). There are indications that ANKRD1 (CARP) is a direct target of TGF-b/Smad signaling and acts as a negative regulator for cell cycle progression. Kanai et al., “Transforming growth factor-beta/Smads signaling induces transcription of the cell type-restricted ankyrin repeat protein CARP gene through CAGA motif in vascular smooth muscle cells,” Circ. Res., 88(1):30-36 (2001). Thus, hHSS1 presumably could be targeting the TGF-b/Smad pathway via ANKRD1 up-regulation. Many genes with direct and indirect relationship with E2F gene were down-regulated by hHSS1. The E2F transcription factor family is known to play a central role in the expression of genes required for cell cycle progression and proliferation, particularly genes involved in DNA synthesis. Stevens et al., “E2F and cell cycle control: a double-edged sword,” Arch. Biochem. Biophys., 412(2):157-169 (2003).

Thus, it is theorized that E2F plays an important role in coordinating events associated with cell cycle arrest mediated by hHSS1. In parallel, hHSS1 regulated genes involved in centrosome separation and maturation (EG5, CDC2, cyclin B), mitotic entry (CDC25, CDC2, Cyclin B, PLK), metaphase and anaphase transition (CDC, APC, PRC1, Cyclin B. Esp1, SMC1), which could also have an effect on cell cycle and consequently cell proliferation. Conversely, hHSS1 overexpression in A172 cells does not seem to regulate a specific cell cycle phase. However, IPA and Ingenuity® iReport pathway analysis of A172 cells indicated that hHSS1 modulated genes related to metabolic pathways, which could in part have an effect over the global protein expression, thereby contributing to the regulation of proliferation. Thus, it is presumed that hHSS1 mechanisms governing cell proliferation in A172 and U87 cells might be different. This difference may be explained based on the dissimilar deletions and genetic mutations linked to these cell lines. Law et al., “Molecular cytogenetic analysis of chromosomes 1 and 19 in glioma cell lines,” Cancer Genet. Cytogenet., 160(1):1-14 (2005).

It is worth noting that IL13RA2 was the most up-regulated gene induced by hHSS1 in U87 cells. The IL13RA2 gene is often overexpressed in brain tumors (Jarboe et al., “Expression of interleukin-13 receptor alpha2 in glioblastoma multiforme: implications for targeted therapies,” Cancer Res., 67(17):7983-7986 (2007)) and is involved in the invasion and metastasis of ovarian cancer cells (Fujisawa et al., “IL-13 regulates cancer invasion and metastasis through IL-13Rα2 via ERK/AP-1 pathway in mouse model of human ovarian cancer.” Int. J. Cancer, 131(2):344-356 (2012)). Overexpression of the IL13RA2 chain in human breast cancer cell line and pancreatic cancer cell line inhibited tumor development in nude mice, probably mediated by IL-13. Kawakami et al., “In vivo overexpression of IL-13 receptor alpha2 chain inhibits tumorigenicity of human breast and pancreatic tumors in immunodeficient,” J. Exp. Med., 194(12):1743-1754 (2001).

IL13RA2 overexpressing tumor cells produced high levels of IL-8 which has been shown to reduce tumorigenicity in several tumor models. Kawakami et al., “In vivo overexpression of IL-13 receptor alpha2 chain inhibits tumorigenicity of human breast and pancreatic tumors in immunodeficient mice,” J. Exp. Med., 194(12):1743-1754 (2001); Lee et al., “IL-8 reduced tumorigenicity of human ovarian cancer in vivo due to neutrophil infiltration,” J. Immunol., 164(5):2769-2775 (2000); and Inoue et al., “Interleukin 8 expression regulates tumorigenicity and metastases in androgen-independent prostate cancer,” Clin. Cancer Res., 6(5):2104-2119 (2000). Decreasing the expression of the IL-13 receptor also leads to an increasing tumorigenicity. Kawakami et al. 2001.

Overexpression of hHSS1 affected the migratory and invasive properties of U87 cells induced by FBS as a chemoattractant. In A172 cells, IPA top molecular network analysis showed that several genes down-regulated by hHSS1 are target genes regulated by VEGF or genes responsible for ERK regulation. However, a consistent negative regulation was not observed in vitro of A172 stable clones migratory or invasive proprieties induced by hHSS1. Variations in migratory and invasive proprieties induced by hHSS1 in different glioma cell lines are likely due to diverse genetic background (e.g. mutations and deletions) (Law et al., “Molecular cytogenetic analysis of chromosomes 1 and 19 in glioma cell lines,” Cancer Genet. Cytogenet., 160(1):1-14 (2005)), probably involving other signaling pathways and molecules.

The data presented herein, however, showed that A172 glioma-derived cells overexpressing hHSS1 significantly inhibited HUVEC migration and invasion in low-serum protein conditions, indicating an indirect functional role for hHSS1 in angiogenesis. Moreover, in the same cell culture conditions, U87 cells overexpressing hHSS1 inhibited invasion but not migration of HUVEC cells. It has been previously reported that stimulation of endothelial cells by tumor cells establishes an endothelial phenotype consistent with the initial stages of angiogenesis. Khodarev et al., “Tumour-endothelium interactions in co-culture: coordinated changes of gene expression profiles and phenotypic properties of endothelial cells,” J. Cell Sci., 116(Pt 6):1013-1022 (2003); and Ferla et al., “Glioblastoma-derived leptin induces tube formation and growth of endothelial cells: comparison with VEGF effects,” BMC Cancer, 11:303 (2011). Although U87-overexpressing hHSS1 cells did not inhibit HUVEC migration, restraint of relevant morphological changes indicative of early angiogenesis were noted in HUVECs that invaded the matrix (i.e. HUVECs did not align themselves to form net-like structures relative to the control cells). Inhibition of net-like formation of HUVEC in co-cultures is consistent with the action of angiogenesis inhibitors like angiostatin and endostatin. Khodarev et al. (2003). Additionally, it was found that treatment of HUVEC cells with purified hHSS1, efficiently inhibited HUVEC tube formation ability, indicating that there is a direct functional relation between hHSS1 and HUVEC cells.

Moreover, the microarray data of U87 glioma cells described herein indicates that hHSS1 down-regulated genes involved in angiogenesis, including THBS1 and APLN. THBS1 is reported to stimulate or inhibit cell adhesion, proliferation, motility and survival in a context-dependent and cell-specific manner. Roberts DD., “Regulation of tumor growth and metastasis by thrombospondin-1,” FASEB 10(10):1183-1191 (1996). Although THBS1 is a potent inhibitor of angiogenesis, N-terminal proteolytic and recombinant peptides related to THBS1 have clear pro-angiogenic activities mediated by beta-1 integrins. Roberts DD., “THBS1 (thrombospondin-1),” Atlas Genet. Cytogenet. Oncol. Haematol., 9(3):231-233 (2005). Moreover, glioma cell lines secrete significant levels of THBS-1, and high levels of THBS1 have been found in glioma tissues. Kawataki et al., “Correlation of thrombospondin-1 and transforming growth factor-beta expression with malignancy of glioma,” Neuropathology, 20(3):161-169 (2000); and Naganuma et al., “Quantification of thrombospondin-1 secretion and expression of alphavbeta3 and alpha3beta1 integrins and syndecan-1 as cell-surface receptors for thrombospondin-1 in malignant glioma cells, Neurooncol., 70(3):309-317 (2004).

Among the most down-regulated genes in U87 is APLN, a ligand for the angiotensin-like 1 (APJ) receptor. O'Dowd et al., “A human gene that shows identity with the gene encoding the angiotensin receptor is located on chromosome 11,” Gene, 136(1-2):355-360 (1993); and Tatemoto et al., “Isolation and characterization of a novel endogenous peptide ligand for the human APJ receptor,” Biochem. Biophys. Res. Commun., 251(2):471-476 (1998). APLN expression has been observed to be highly up-regulated in the microvasculature in brain tumors. In particular, APLN has been shown to be needed for intersomitic vessel angiogenesis and the promotion of angiogenesis in brain tumors. Kälin et al., “Paracrine and autocrine mechanisms of apelin signaling govern embryonic and tumor angiogenesis,” Dev. Biol., 305(2):599-614 (2007).

It is of further interest that ADAMTS5 was among the highly up-regulated genes. ADAMTS5 is a metalloproteinase with the ability to slow tumor growth and diminish tumor angiogenesis, together with reduced tumor cell proliferation and increased tumor cell apoptosis. Kumar et al., “ADAMTS5 functions as an anti-angiogenic and anti-tumorigenic protein independent of its proteoglycanase activity,” Am. J. Pathol., 181(3):1056-1068 (2012). The fact that hHSS1 strongly down-regulates THBS-1 and APLN, and highly up-regulates ADAMTS5 in the hHSS1-overexpressing cells is consistent with the observed in vitro results where angiogenesis was greatly suppressed by purified hHSS1. It is important to note that the GBM TCGA database analysis did not show a significant correlation between hHSS1 and the expression of APLN and THBS-1 genes, as observed for the microarray analysis using U87 hHSS1-overexpressing cells. This discrepancy could be due to potentially lower expression levels of hHSS1 in tumor tissues (not higher than 3.5-fold relative to normalization controls) compared to U87 cells ectopically overexpressing hHSS1 (11.7-fold). In addition, most of the 12 genes evaluated were expressed in the tumor tissue at relatively lower levels than 3.5-fold.

It was recently suggested that BRCA1-2 carriers present higher expression of angiogenic factors VEGF, HIF-1a and higher microvessel density than in sporadic cancers (Saponaro et al., “HIF-1α expression and MVD as an angiogenic network in familial breast cancer,” PLoS One, 8(1):e53070 (2013)), thus providing a link between BRCA genes and angiogenesis. Interestingly, the analysis of GBM dataset from TCGA revealed a highly significant inverse correlation between hHSS1 and BRCA2 expression, and that the levels of BRCA2 expression on HSS1-high gliomas were also significantly lower than on HSS1-low expression gliomas. This finding is intriguing in light of tube formation data that suggested purified hHSS1 inhibits HUVEC tube formation, thus implicating a role of hHSS1 in angiogenesis. It has been shown that BRCA2-defective cancer cells or treatment of cancer cells with BRCA2 siRNA significantly reduces BRCA2 protein and mRNA expression, leading to tumor radio-sensitization in vitro and in vivo, mainly through the inhibition of homologous recombination repair. Dong et al., “Down regulation of BRCA2 causes radio-sensitization of human tumor cells in vitro and in vivo,” Cancer Sci., 99(4):810-815 (2008); and Abbott et al., “Double-strand break repair deficiency and radiation sensitivity in BRCA2 mutant cancer cells,” J. Natl. Cancer Inst., 90(13):978-985 (1998). Moreover, knockdown of BRCA2 greatly sensitizes glioma cells to DNA double strand breaks and the induction of cell death following temozolomide and nimustine treatment. Quiros et al., “Rad51 and BRCA2-New molecular targets for sensitizing glioma cells to alkylating anticancer drugs,” PLoS One, 6(11):e27183 (2011).

ADAMTS1 is a protease commonly up-regulated in metastatic carcinoma. ADAMTS1 processing of versican is important in cell migration during wound healing and endothelial cell invasion. Krampert et al., “ADAMTS1 proteinase is up-regulated in wounded skin and regulates migration of fibroblasts and endothelial cells,” J. Biol. Chem., 280(25):23844-23852 (2005); and Su et al., “Molecular profile of endothelial invasion of three-dimensional collagen matrices: insights into angiogenic sprout induction in wound healing,” Am. J. Physiol. Cell Physiol., 295(5):C1215-C1229 (2008). In addition, up-regulation of ADAMTS1 in tumors participate in the remodeling of the peritumoral stroma, tumor growth and metastasis. Ricciardelli et al., “The ADAMTS1 protease gene is required for mammary tumor growth and metastasis,” Am. J. Pathol., 179(6):3075-3085 (2011).

The analysis from the TCGA database described herein suggests a significant inverse correlation between hHSS1 and ADAMTS1 expression, which is consistent with a role of hHSS1 in inhibition of tumor growth, progression and metastasis. GBM from TCGA also revealed a significant positive correlation between hHSS1 and endostatin (COL18A1) expression. Endogenous expression of endostatin by C6 glioma cells result in a reduced tumor growth rate in vivo that is associated with inhibition of tumor angiogenesis. Peroulis et al., “Antiangiogenic activity of endostatin inhibits C6 glioma growth,” Int. J. Cancer, 97(6):839-845 (2002).

The data described herein suggests that hHSS1 can be used as an adjuvant therapy for the effective treatment of gliomas.

It was reported that endostatin blocks VEGF-induced tyrosine phosphorylation of KDR/Flk-1 and activation of ERK, p38 MAPK, and p125FAK in human umbilical vein endothelial cells. Kim et al., “Endostatin blocks vascular endothelial growth factor mediated signaling via direct interaction with KDR/Flk-1,” J. Biol. Chem., 31:27872-27879 (2002). IPA top molecular network analysis in A172 cells showed that several genes down-regulated by hHSS1 are target genes regulated by VEGF or genes responsible for ERK regulation. Development of endostatin has been undertaken for the treatment of gliomas based on extensive preclinical data. Grossman et al., “Improvement in the standard treatment for experimental glioma by fusing antibody Fc domain to endostatin,” J. Neurosurg., 115(6):1139-1146 (2011). The mechanism of action focused on inhibition of angiogenesis highlights the possibility of combining hHSS1 and endostatin in the potential treatment of glioma. A potential synergistic effect could also lead to dose reductions in the level of administered therapeutic agent.

Angiogenesis is a complex process that involves the activation, proliferation, migration and invasion of endothelial cells to form new capillaries from existing blood vessels. The endothelial cells involved in tumor development dissolve their surrounding extracellular matrix, migrate toward the tumor, proliferate and form a new vascular network. Oklu et al., “Angiogenesis and current antiangiogenic strategies for the treatment of cancer,” J. Vase. Interv. Radiol., 21(12):1791-1805 (2010). The anti-angiogenic effect of hHSS1 seems to correlate with the effect of the potent angiogenesis inhibitor endostatin (Rosca et al., “Anti-angiogenic peptides for cancer therapeutics,” Curr. Pharm. Biotechnol., 12(8):1101-1116 (2011)), in that both proteins are extracellular proteins with the ability to negatively regulate HUVEC cell migration, invasion, tube formation as well as invasion of tumor cells. Kim et al. (2000).

### III. Methods of Treatment

A. Cancer Treatment Methods

The present invention is directed to methods of treating cancer comprising administering a therapeutically effective amount of HSS1 or human hHSS1, HSM1 or human hHSM1, or a combination thereof; as either a solo therapy or as an adjuvant to a conventional cancer therapy used now or later discovered. The subject is preferably a mammal, including, but not limited to, animals such as cows, pigs, horses, chickens, cats, dogs, etc., and is most preferably human.

Included in the definition of “conventional cancer therapies” are all forms of radiation therapy and all forms of chemotherapies, which can be used in conjunction with various forms of radiation therapy. These conventional cancer therapies also include surgery to remove all or part of the cancerous tissue, along with any combination of radiation and chemotherapy.

In another embodiment, the cancer patient population to be treated is a carrier of BRCA1-2.

When used as an adjuvant to chemotherapy, radiation therapy, or surgery, HSS1 and/or HSM1 can be administered before, during, or after chemotherapy, radiation therapy, surgery, or any combination of before, during, or after chemotherapy, radiation therapy, and/or surgery.

The dose of HSS1 and/or HSM1 used in the present invention is the dose required to be efficacious as well as safe, regardless of how HSS1 and/or HSM1 is delivered.

In yet another embodiment of the invention, HSS1 and/or HSM1 can inhibit tumor angiogenesis.

In yet another embodiment of the invention. HSS1 and/or HSM1 can radiosensitize a tumor or cancer, which makes tumor or cancer cells more susceptible to radiation therapy, leading to more effective radiation treatment of the tumor or cancer. For example, in one embodiment of the invention, administration of HSS1 and/or HSM1, when used in combination with radiation therapy, radiosensitizes the tumor or cancer resulting in an improvement of therapy by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The improvement in therapy can be measured by, for example, (1) a decrease in tumor size; (2) decrease in tumor progression, (3) decrease in metastasis, or (4) any combination thereof.

The methods and compositions of the invention can increase the survival of patients diagnosed with cancer. For example, in one embodiment of the invention, administration of HSS1 and/or HSM1, when used in combination either alone or in combination with a cancer therapy, increases survival by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as compared to the survival rate of a cancer patient population receiving the same cancer therapy (or no cancer therapy) but in the absence of an HSS1 and/or HSM1 composition.

Other benefits of the methods and compositions of the invention in treating cancer patients include a reduction in tumor mass, more complete cancer remission, and/or slowing or inhibition of tumor growth, progression, and/or metastasis. The improvement, as measured by decreased tumor mass, more complete cancer remission, slowing or inhibition of tumor growth, slowing or inhibition of tumor progression, and/or slowing or inhibition of tumor metastasis, can be by 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as compared to the same indicia (e.g., tumor growth, progression, etc.) of a cancer patient population receiving the same cancer therapy (or no cancer therapy) but in the absence of an HSS1 and/or HSM1 composition.

B. Inflammatory Disease Treatment Methods

In another embodiment of the invention encompassed are methods for treating inflammatory diseases by reducing inflammatory cell invasion by anti-angiogenic activity, comprising administering to a subject in need a composition according to ther invention.

It is known that angiogenesis has a role in many inflammatory diseases. The perpetuation of neovascularization in inflammatory diseases, such as rheumatoid arthritis, spondyloarthropathies and some systemic autoimmune diseases, might facilitate the ingress of inflammatory cells into the synovium and, therefore, stimulate pannus formation. Disorders associated with perpetuated neovascularization are considered to be angiogenic inflammatory diseases. Furthermore, angiogenesis might be targeted by several specific approaches that could be therapeutically used to control inflammatory diseases. Szekanecz et al., Nat. Clin. Pract. Rheumatol., 3(11):635-43 (2007).

Exemplary inflammatory diseases that can be treated with compositions according to the invention include any known inflammatory disease, such as but not limited to Arthritis, rheumatoid arthritis (RA), Crohn's disease, Psoriasis, Endometriosis, chronic inflammatory diseases (e.g., inflammatory bowel disease (IBD)), osteoarthritis, asthma, pulmonary fibrosis, celiac disease, vasculitis, lupus, chronic obstructive pulmonary disease (COPD), pelvic inflammatory disease, chronic peptic ulcer, tuberculosis, chronic periodontitis, ulcerative colitis, chronic sinusitis, and chronic active hepatitis.

C. Modes of Administration

Any pharmaceutically acceptable delivery method now known or developed in the future can be utilized in the methods of the invention to deliver HSS1 and/or HSM1 to the cancer patient, either locally or systemically. Various delivery systems are known and can be used to administer HSS1 and/or HSM1 in accordance with the methods of the invention, e.g., encapsulation in liposomes, microparticles, microcapsules, recombinant cells capable of expressing HSS1 and/or HSM1, receptor-mediated endocytosis, construction of nucleic acid comprising a gene for HSS1 and/or HSM1 as part of a retroviral or other vector, etc.

In one aspect, the one or more effective doses of HSS1 and/or HSM1 are administered topically, orally, via an epidural, intranasaly, subcutaneously, intradermally, intravenously, intraperitoneally, intramuscularly, epidurally, parenterally, intranasally, and/or intracranially.

Thus, for example, HSS1 and/or HSM1 can be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce pharmaceutical compositions comprising HSS1 and/or HSM1 into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. It may be desirable to administer the pharmaceutical compositions comprising HSS1 and/or HSM1 locally to the area in need of treatment; this may be achieved, for example and not by way of limitation, by topical application, by injection, by means of a catheter, by means of a suppository, or by means of an implant, said implant being of a porous, non-porous, or gelatinous material, including membranes, such as sialastic membranes, or fibers.

Still other modes of administration of HSS1 and/or HSM1 involve delivery in a controlled release system. In certain embodiments, a pump may be used. Additionally polymeric materials can be used, or a controlled release system can be placed in proximity of the therapeutic target, thus requiring only a fraction of the systemic dose.

Another form of delivery method for the HSS1 and/or HSM1 is via various gene therapy vector delivery systems available in the art or to be discovered in the future. In one embodiment of the invention, the gene therapy vector is derived from adenovirus. In another embodiment of the invention, the gene therapy vector is derived from the herpes virus. In still another embodiment of the invention, the gene therapy vector is derived from a retrovirus.

Further, if a brain cancer is to be treated, any methods now known or developed in the future that facilitate passage across the blood brain barrier can be utilized in the methods of the invention to deliver HSS1 and/or HSM1 to the site of the brain cancer (although local delivery to the brain cancer is not required). When a brain cancer is to be treated, HSS1 and/or HSM1 can be formulated as a pharmaceutical for systemic delivery or for delivery to the brain by intracerebroventricular infusion, or any other like delivery method.

A. Exemplary Cancers Treatable Using the Methods and Compositions of the Invention

1. Brain Cancer

In one embodiment of the invention, the cancer to be treated is a brain cancer. In this embodiment, HSS1 and/or HSM1 are used as an adjuvant to a conventional cancer therapy.

The brain cancer can be a primary or secondary brain cancer. The preferred brain cancer treatable with embodiments of the present invention is glioma, particularly glioblastoma multiforme. Other brain cancers are also treatable with the present invention, including but not limited to astrocytoma, oligodendroglioma, ependymoma, meningiomas, neuroblastoma, acoustic neuroma/schwannomas, and medulloblastoma.

In one embodiment of the invention, the brain cancer to be treated with a method of the invention is a secondary brain cancer which has metastatized from a non-brain cancer.

Exemplary types of brain cancer treatable with the methods and compositions according to the invention include the following. (1) Gliomas. These tumors occur in the glial cells, which help support and protect critical areas of the brain. Gliomas are the most common type of brain tumor in adults, responsible for about 42% of all adult brain tumors. Gliomas are further characterized by the types of cells they affect. (2) Astrocytoma: Astrocytes are star-shaped cells that protect neurons. Tumors of these cells can spread from the primary site to other areas of the brain, but rarely spread outside the central nervous system. Astrocytomas are graded from I to IV depending on the speed of progression: (i) Grade I (pilocytic astrocytoma): slow growing, with little tendency to infiltrate surrounding brain tissue. Most common in children and adolescents; (ii) Grade II (diffuse astrocytoma): fairly slow-growing, with some tendency to infiltrate surrounding brain tissue. Mostly seen in young adults; (iii) Grade In (anaplastic/malignant astrocytoma): these tumors grow rather quickly and infiltrate surrounding brain tissue; and (iv) Grade IV (glioblastoma multiforme, GBM): an extremely aggressive and lethal form of brain cancer. Unfortunately, it is the most common form of brain tumor in adults, accounting for 67% of all astrocytomas. (3) Oligodendroglioma: Oligodendrocytes are cells that make myelin, a fatty substance that forms a protective sheath around nerve cells. Oligodendrogliomas, which make up 4% of brain tumors, mostly affect people over 45 years of age. Some subtypes of this tumor are particularly sensitive to treatment with radiation therapy and chemotherapy. Half of patients with oligodendrogliomas are still alive after five years. (4) Ependymoma: These tumors affect ependymal cells, which line the pathways that carry cerebrospinal fluid throughout the brain and spinal cord. Ependymomas are rare; about 2% of all brain tumors, but are the most common brain tumor in children. They generally do not affect healthy brain tissue and do not spread beyond the ependyma. Although these tumors respond well to surgery, particularly those on the spine, ependymomas cannot always be completely removed. The five-year survival rate for patients over age 45 approaches 70%. (5) Meningiomas: These tumors affect the meninges, the tissue that forms the protective outer covering of the brain and spine. One-quarter of all brain and spinal tumors are meningiomas, and up to 85% of them are benign. Meningiomas can occur at any age, but the incidence increases significantly in people over age 65. Women are twice as likely as men to have meningiomas. They generally grow very slowly and often don't produce any symptoms. In fact, many meningiomas are discovered by accident. Meningiomas can be successfully treated with surgery, but some patients, particularly the elderly, may be candidates for watchful waiting to monitor the disease. (6) Acoustic Neuroma/Schwannomas: Schwann's cells are found in the sheath that covers nerve cells. Vestibular schwannomas, also known as acoustic neuromas, arise from the 8th cranial nerve, which is responsible for hearing. Specific symptoms of vestibular schwannoma include buzzing or ringing in the ears, one-sided hearing loss and/or balance problems. Schwannomas are typically benign and respond well to surgery. (7) Medulloblastoma: Medulloblastoma is a common brain tumor in children, usually diagnosed before the age of 10. These tumors occur in the cerebellum, which has a crucial role in coordinating muscular movements. Some experts believe that medulloblastomas arise from fetal cells that remain in the cerebellum after birth. Tumors grow quickly and can invade neighboring portions of the brain, as well as spreading outside the central nervous system. Medulloblastoma is slightly more common in boys.

2. Non-Brain Cancer

In another embodiment of the invention, HSS1 and/or HSM1 are used as a solo antiangiogenic cancer therapy, or as an adjuvant to a conventional cancer therapy, wherein the cancer is not a brain cancer.

Any non-brain cancer can be treated using the compositions and methods of the invention in either a solo or combination therapy. In one embodiment, the non-brain cancer is ovarian cancer, pancreatic cancer, or breast cancer.

The cancer subject to be treated can have any type of cancer, including but not limited to a solid tumor type of cancer, a non-solid tumor type of cancer, a hematopoietic cancer, or a leukemia. Preferred non-solid tumor cancers treatable with the methods of the invention include but are not limited to leukemias. In addition, examples of types of cancer treatable with the methods of the invention include but are not limited to, a solid tumor, carcinomas, sarcomas, lymphomas, cancers that begin in the skin, and cancers that begin in tissues that line or cover internal organs. In another embodiment, examples of such types of cancer include, but are not limited to, leukemias, lymphomas, thyroid cancer, head and neck cancer, skin cancer, including melanoma, kidney cancer, gastrointestinal cancers, cancer of the digestive system, esophageal cancer, gallbladder cancer, liver cancer, pancreatic cancer, stomach cancer, small intestine cancer, large intestine (colon) cancer, rectal cancer, gynecological cancers, cervical cancer, ovarian cancer, uterine cancer, vaginal cancer, vulvar cancer, prostate cancer, bladder cancer, endometrial cancer, breast cancer, and lung cancer.

B. Exemplary Combination

Therapy with HSS1 and/or HSM1

The HSS1 and/or HSM1 compositions of the invention can be combined with any useful cancer therapy in treating a patient according to the methods of the invention. By “combined,” it is meant that the HSS1 and/or HSM1 compositions of the invention can be co-administered with a cancer treatment, or the compositions of the invention can be administered before, during, or after a cancer treatment. For example, the compositions of the invention can be administered “near the time of administration of the cancer treatment”, meaning the administration of HSS1 and/or HSM1 at any reasonable time period either before, during, and/or after the administration of the cancer treatment, such as about one month, about three weeks, about two weeks, about one week, several days, about 120 hours, about 96 hours, about 72 hours, about 48 hours, about 24 hours, about 20 hours, several hours, about one hour or minutes before or after administration of the cancer treatment.

Exemplary active agents used to treat cancers include, but are not limited to, angiogenesis inhibitors. Examples of endogenous angiogenesis inhibitors include, but are not limited to, soluble VEGFR-1 and NRP-1, Angiopoietin 2, TSP-1 and TSP-2, angiostatin and related molecules, endostatin, vasostatin, calreticulin, platelet factor-4, TIMP and CDAI, Meth-1 and Meth-2, IFN-α, -β and -γ, CXCL10, IL-4, IL-12 and IL-18, prothrombin (kringle domain-2), antithrombin III fragment, prolactin, VEGI, SPARC, osteopontin, maspin, canstatin (a fragment of COL4A2), and proliferin-related protein. Examples of exogenous angiogenesis inhibitors (e.g., drugs) include but are not limited to, bevacizumab, itraconazole, carboxyamidotriazole, TNP-470 (an analog of fumagillin), CM101, IFN-α, IL-12, platelet factor-4, suramin, SU5416, Thrombospondin, VEGFR antagonists, angiostatic steroids+heparin, Cartilage-Derived Angiogenesis Inhibitory Factor, matrix metalloproteinase inhibitors, angiostatin, endostatin, 2-methoxyestradiol, tecogalan, tetrathiomolybdate, thalidomide, thrombospondin, prolactin, αVβ3 inhibitors, linomide, tasquinimod, ranibizumab, sorafenib (Nexavar®), sunitinib (Sutent®), pazopanib (Votrient®), and everolimus (Afinitor®).

Combination with chemotherapy: In one aspect, the aspects and embodiments of the present disclosure can be utilized as a combined therapy with existing chemotherapeutic modalities. The combination (sequential or concurrent) therapy can be co-administration or co-formulation. “Chemotherapy” refers to any therapy that includes natural or synthetic agents now known or to be developed in the medical arts. Examples of chemotherapy include the numerous cancer drugs that are currently available. However, chemotherapy also includes any drug, natural or synthetic, that is intended to treat a disease state. In certain embodiments of the invention, chemotherapy may include the administration of several state of the art drugs intended to treat the disease state. Examples include combined chemotherapy with docetaxel, cisplatin, and 5-fluorouracil for patients with locally advanced squamous cell carcinoma of the head, and fludarabine and bendamustine in refractory and relapsed indolent lymphoma.

The HSS1 and/or HSM1 compositions of the invention can also be used in combination with radiation therapy to treat a cancer patient. “Radiation therapy” refers to any therapy where any form of radiation is used to treat the disease state. The instruments that produce the radiation for the radiation therapy are either those instruments currently available or to be available in the future.

### IV. Compositions

The invention encompasses pharmaceutical compositions useful in the methods of the invention. The compositions comprise HSS1, HSM1, or a combination thereof. The compositions can additionally comprise at least one pharmaceutically acceptable carrier. In another embodiment, the pharmaceutical compositions can additionally comprise at least one active agent which is not HSS1 or HSM1, wherein the active agent is useful in treating a cancer.

The HSS1 and/or HSM1 molecule may be present in a substantially isolated form. It will be understood that the product may be mixed with carriers or diluents which will not interfere with the intended purpose of the product and still be regarded as substantially isolated. A product of the invention may also be in a substantially purified form, in which case it will generally comprise about 80%, 85%, or 90%, including, for example, at least about 95%, at least about 98% or at least about 99% of the peptide or dry mass of the preparation.

Generally, the amino acid sequences of the HSS1 and/or HSM1 molecule used in embodiments of the invention are derived from the specific mammal to be treated by the methods of the invention. Thus, for the sake of illustration, for humans, generally human HSS1 and/or HSM1, or recombinant human HSS1 and/or HSM1, would be administered to a human in the methods of the invention, and similarly, for felines, for example, the feline HSS1 and/or HSM1, or recombinant feline HSS1 and/or HSM1, would be administered to a feline in the methods of the invention.

Also included in the invention, however, are certain embodiments where the HSS1 and/or HSM1 molecule does not derive its amino acid sequence from the mammal that is the subject of the therapeutic methods of the invention. For the sake of illustration, human HSS1 and/or HSM1 or recombinant human HSS1 and/or HSM1 may be utilized in a feline mammal. Still other embodiments of the invention include HSS1 and/or HSM1 molecules where the native amino acid sequence of HSS1 and/or HSM1 is altered from the native sequence, but the HSS1 and/or HSM1 molecule functions to yield the anti-cancer properties of HSS1 and/or HSM1 that are disclosed herein. Alterations from the native, species-specific amino acid sequence of HSS1 and/or HSM1 include changes in the primary sequence of HSS1 and/or HSM1 and encompass deletions and additions to the primary amino acid sequence to yield variant HSS1 and/or HSM1 molecules. Also included are modified HSS1 and/or HSM1 molecules are also included in the methods of invention, such as covalent modifications to the HSS1 and/or HSM1 molecule that increase its shelf life, half-life, potency, solubility, delivery, etc., additions of polyethylene glycol groups. Other HSS1 and/or HSM1 variants included in the present disclosure are those where the canonical sequence is post-translationally-modified, for example, glycosylated.

In another embodiment, the compositions comprise (I) a peptide having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to HSS1, (2) a peptide having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to HSM1, or (3) any combination thereof. In addition, the invention encompasses compositions comprising at least one HSS1 fragment, HSM1 fragment, or a combination of at least one HSS1 fragment and at least one HSM1 fragment. Thus, the invention encompasses pharmaceutical compositions comprising a therapeutically effective amount of HSS1, HSM1, at least one HSS1 fragment, at least one HSM1 fragment, a peptide having at least about 80% homology to HSS1 (or a homology as defined herein), a peptide having at least about 80% homology to HSM1 (or a homology as defined herein), or any combination thereof.

The terms “HSS1 fragment” and “HSM1 fragment” refer to a peptide that has an amino-terminal and/or carboxy-terminal deletion as compared to the native protein, but where the remaining amino acid sequence is identical to the corresponding positions in the amino acid sequence deduced from a full-length cDNA sequence. Fragments typically are at least about 4 amino acids in length. The full-length cDNA sequence of HSS1 consists of approximately 1.9 kb containing an open reading frame of 789 bp (e.g., corresponding to about 263 amino acids). In other embodiments of the invention, the HSS1 fragment and/or HSM1 fragment has a size of about 5 amino acids, about 6 amino acids, about 7 amino acids, about 8 amino acids, about 9 amino acids, about 10 amino acids, about 11 amino acids, about 12 amino acids, about 13 amino acids, about 14 amino acids, about 15 amino acids, about 16 amino acids, about 17 amino acids, about 18 amino acids, about 19 amino acids, about 20 amino acids, about 25 amino acids, about 30 amino acids, about 35 amino acids, about 40 amino acids, about 45 amino acids, about 50 amino acids, about 55 amino acids, about 60 amino acids, about 65 amino acids, about 70 amino acids, about 75 amino acids, about 80 amino acids, about 85 amino acids, about 90 amino acids, about 95 amino acids, about 100 amino acids, about 105 amino acids, about 110 amino acids, about 115 amino acids, about 120 amino acids, about 125 amino acids, about 130 amino acids, about 135 amino acids, about 140 amino acids, about 145 amino acids, about 150 amino acids, about 155 amino acids, about 160 amino acids, about 165 amino acids, about 170 amino acids, about 175 amino acids, about 180 amino acids, about 185 amino acids, about 190 amino acids, about 195 amino acids, about 200 amino acids, about 205 amino acids, about 210 amino acids, about 215 amino acids, about 220 amino acids, about 225 amino acids, about 230 amino acids, about 235 amino acids, about 240 amino acids, about 245 amino acids, about 250 amino acids, about 255 amino acids, or about 260 amino acids. Preferably, the fragment spans at least one epitope of the full-length HSS1 or HSM1.

Since it is often difficult to predict in advance the characteristics of a variant HSS1 and/or HSM1 polypeptide, it will be appreciated that some screening of the recovered variant will be needed to select the optimal variant, e.g., to confirm that the variant exhibits anti-angiogenic activity.

Dosage Forms:

The pharmaceutical compositions may be formulated for immediate release, sustained release, controlled release, delayed release, or any combinations thereof. The pharmaceutical compositions for administration may be administered in a single administration or in multiple administrations.

Suitable dosage forms of HSS1 and/or HSM1 for use in embodiments of the present invention encompass physiologically acceptable carriers that are inherently non-toxic and non-therapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and PEG. Carriers for topical or gel-based forms of HSS1 and/or HSM1 polypeptides include polysaccharides such as sodium carboxymethylcellulose or methylcellulose, polyvinylpyrrolidone, polyacrylates, polyoxyethylene-polyoxypropylene-block polymers, PEG, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations.

Suitable examples of sustained-release preparations include semipermeable matrices of solid hydrophobic polymers containing the polypeptide, which matrices are in the form of shaped articles, e.g. films, or microcapsules. Examples of sustained-release matrices include polyesters, hydrogels (for example, poly(2-hydroxyethyl-methacrylate), or poly(vinylalcohol), polylactides, copolymers of L-glutamic acid and gamma ethyl-L-glutamate, non-degradable ethylene-vinyl acetate, degradable lactic acid-glycolic acid copolymers such as the Lupron Depot™ (injectable microspheres composed of lactic acid-glycolicacid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated HSS1 and/or HSM1 polypeptides remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Sustained-release HSS1 and/or HSM1 containing compositions also include liposomally entrapped polypeptides. Liposomes containing a HSS1 and/or HSM1 polypeptide are prepared by methods known in the art. Ordinarily, the liposomes are the small (about 200-800 Angstroms) unilamelar type in which the lipid content is greater than about 30 mol. % cholesterol, the selected proportion being adjusted for the optimal Wnt polypeptide therapy. Liposomes with enhanced circulation time are disclosed in U.S. Pat. No. 5,013,556.

For the treatment of disease, the appropriate dosage of a HSS1 and/or HSM1 polypeptide will depend on the type of disease to be treated, as defined above, the severity and course of the disease, previous therapy, the patient's clinical history and response to the HSS1 and/or HSM1 therapeutic methods disclosed herein, and the discretion of the attending physician. In accordance with the invention, HSS1 and/or HSM1 is suitably administered to the patient at one time or over a series of treatments.

Therapeutic formulations of HSS1 and/or HSM1 are prepared for storage by mixing HSS1 and/or HSM1 having the desired degree of purity with optional physiologically acceptable carriers, excipients, or stabilizers (Remington's Pharmaceutical Sciences, 16th edition, Osol, A., Ed., (1980)), in the form of lyophilized cake or aqueous solutions. Acceptable carriers, excipients, or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counter-ions such as sodium; and/or nonionic surfactants such as Tween®, Pluronics™ or polyethylene glycol (PEG).

The term “buffer” as used herein denotes a pharmaceutically acceptable excipient, which stabilizes the pH of a pharmaceutical preparation. Suitable buffers are well known in the art and can be found in the literature. Pharmaceutically acceptable buffers include but are not limited to histidine-buffers, citrate-buffers, succinate-buffers, acetate-buffers, phosphate-buffers, arginine-buffers or mixtures thereof. The abovementioned buffers are generally used in an amount of about 1 mM to about 100 mM, of about 5 mM to about 50 mM and of about 10-20 mM. The pH of the buffered solution can be at least 4.0, at least 4.5, at least 5.0, at least 5.5 or at least 6.0. The pH of the buffered solution can be less than 7.5, less than 7.0, or less than 6.5. The pH of the buffered solution can be about 4.0 to about 7.5, about 5.5 to about 7.5, about 5.0 to about 6.5, and about 5.5 to about 6.5 with an acid or a base known in the art, e.g. hydrochloric acid, acetic acid, phosphoric acid, sulfuric acid and citric acid, sodium hydroxide and potassium hydroxide. As used herein when describing pH, “about” means plus or minus 0.2 pH units.

As used herein, the term “surfactant” can include a pharmaceutically acceptable excipient which is used to protect protein formulations against mechanical stresses like agitation and shearing. Examples of pharmaceutically acceptable surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulphate (SDS). Suitable surfactants include polyoxyethylenesorbitan-fatty acid esters such as polysorbate 20, (sold under the trademark Tween 20®) and polysorbate 80 (sold under the trademark Tween 80®). Suitable polyethylene-polypropylene copolymers are those sold under the names Pluronic® F68 or Poloxamer 188®. Suitable Polyoxyethylene alkyl ethers are those sold under the trademark Brij®. Suitable alkylphenolpolyoxyethylene esthers are sold under the trade name Triton-X. When polysorbate 20 (Tween 20®) and polysorbate 80 (Tween 80®) are used they are generally used in a concentration range of about 0.001 to about 1%, of about 0.005 to about 0.2% and of about 0.01% to about 0.1% w/v (weight/volume).

As used herein, the term “stabilizer” can include a pharmaceutical acceptable excipient, which protects the active pharmaceutical ingredient and/or the formulation from chemical and/or physical degradation during manufacturing, storage and application. Chemical and physical degradation pathways of protein pharmaceuticals are reviewed by Cleland et al., Crit. Rev. Ther. Drug Carrier Syst., 70(4):307-77 (1993); Wang, Int. J. Pharm., 7S5(2): 129-88 (1999); Wang, Int. J. Pharm., 203(1-2): 1-60 (2000); and Chi et al, Pharm. Res., 20(9): 1325-36 (2003). Stabilizers include but are not limited to sugars, amino acids, polyols, cyclodextrines, e.g. hydroxypropyl-beta-cyclodextrine, sulfobutylethyl-beta-cyclodextrin, beta-cyclodextrin, polyethylenglycols, e.g. PEG 3000, PEG 3350, PEG 4000, PEG 6000, albumine, human serum albumin (HSA), bovine serum albumin (BSA), salts, e.g. sodium chloride, magnesium chloride, calcium chloride, chelators, e.g. EDTA as hereafter defined. As mentioned hereinabove, stabilizers can be present in the formulation in an amount of about 10 to about 500 mM, an amount of about 10 to about 300 mM, or in an amount of about 100 mM to about 300 mM. In some embodiments, exemplary HSS1 and/or HSM1 can be dissolved in an appropriate pharmaceutical formulation wherein it is stable.

HSS1 and/or HSM1 to be used for in viva administration must be sterile. This is readily accomplished by filtration through sterile filtration membranes, prior to or following lyophilization and reconstitution. HSS1 and/or HSM1 ordinarily will be stored in lyophilized form or in solution. Therapeutic HSS1 and/or HSM1 compositions generally are placed into a container having a sterile access port, for example, an intravenous solution bag or vial having a stopper pierceable by a hypodermic injection needle.

When applied topically, HSS1 and/or HSM1 is suitably combined with other ingredients, such as carriers and/or adjuvants. There are no limitations on the nature of such other ingredients, except that they must be physiologically acceptable and efficacious for their intended administration, and cannot degrade the activity of the active ingredients of the composition. Examples of suitable vehicles include ointments, creams, gels, or suspensions, with or without purified collagen. The compositions also may be impregnated into transdermal patches, plasters, and bandages, preferably in liquid or semi-liquid form.

For obtaining a gel formulation, HSS1 and/or HSM1 formulated in a liquid composition may be mixed with an effective amount of a water-soluble polysaccharide or synthetic polymer such as PEG to form a gel of the proper viscosity to be applied topically. The polysaccharide that may be used includes, for example, cellulose derivatives such as etherified cellulose derivatives, including alkyl celluloses, hydroxyalkyl celluloses, and alkylhydroxyalkyl celluloses, for example, methylcellulose, hydroxyethyl cellulose, carboxymethyl cellulose, hydroxypropyl methylcellulose, and hydroxypropyl cellulose; starch and fractionated starch; agar; alginic acid and alginates; gum arabic; pullullan; agarose; carrageenan; dextrans; dextrins; fructans; inulin; mannans; xylans; arabinans; chitosans; glycogens; glucans; and synthetic biopolymers; as well as gums such as xanthan gum; guar gum; locust bean gum; gum arabic; tragacanth gum; and karaya gum; and derivatives and mixtures thereof. The preferred gelling agent herein is one that is inert to biological systems, nontoxic, simple to prepare, and not too runny or viscous, and will not destabilize the HSS1 and/or HSM1 molecule held within it.

### V. Definitions

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

As used herein, except where the context requires otherwise, the term “comprise” and variations of the term, such as “comprising,” “comprises” and “comprised” are not intended to exclude other additives, components, integers or steps.

“Disease state” refers to a condition present in a mammal whereby the health and well-being of the mammal is compromised. In the present invention, various forms of cancer are the targeted disease states of the invention. In certain embodiments of the invention, treatments intended to target the disease state are administered to the mammal.

“A treatment” is intended to target the disease state and combat it, i.e., ameliorate the disease state. The particular treatment thus will depend on the disease state to be targeted and the current or future state of medicinal therapies and therapeutic approaches. A treatment may have associated toxicities.

“Chemotherapy” refers to any therapy that includes natural or synthetic agents now known or to be developed in the medical arts. Examples of chemotherapy include the numerous cancer drugs that are currently available. However, chemotherapy also includes any drug, natural or synthetic, that is intended to treat a disease state. In certain embodiments of the invention, chemotherapy may include the administration of several state of the art drugs intended to treat the disease state. Examples include combined chemotherapy with docetaxel, cisplatin, and 5-fluorouracil for patients with locally advanced squamous cell carcinoma of the head (Tsukuda et al., Int. J. Clin. Oncol., 9(3):161-6 (June 2004)), and fludarabine and bendamustine in refractory and relapsed indolent lymphoma (Konigsmann et al., Leuk. Lymphoma, 45(9):1821-1827 (2004)).

“Radiation or radiation therapy or radiation treatment” refers to any therapy where any form of radiation is used to treat the disease state. The instruments that produce the radiation for the radiation therapy are either those instruments currently available or to be available in the future.

“Solid tumors” generally is manifested in various cancers of body tissues, such as those solid tumors manifested in lung, breast, prostate, ovary, etc., and are cancers other than cancers of blood tissue, bone marrow or the lymphatic system.

“Hematopoietic disorders (cancers)” generally refers to the presence of cancers of the hematopoietic system such, as leukemias, lymphomas etc.

“Hematopoietic stem cells” are generally the blood stem cells; there are two types: “long-term repopulating” as defined above, and “short-term repopulating” which can produce “progenitor cells” for a short period (weeks, months or even sometimes years depending on the mammal); these are also referred to herein as hematopoietic repopulating cells.

“Hematopoietic progenitor cells” are generally the first cells to differentiate from (i.e., mature from) blood stem cells; they then differentiate (mature) into the various blood cell types and lineages.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, apes, and prenatal, pediatric, and adult humans.

The term “one or more therapeutically effective dose(s) of HSS1 and/or HSM1” refers to any dose administered for any time intervals and for any duration that produce the desired therapeutic effect.

The term “therapeutically effective amount or dose” is defined herein as a dose of a substance that produces effects for which it is administered. The exact dose of HSS1 and/or HSM1 will depend on the purpose of the treatment, the timing of administration of HSS1 and/or HSM1, certain characteristics of the subject to be treated, and the severity of the cancer, and is ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20^{th} Edition, 2003, Gennaro, Ed., Lippincott, Williams & Wilkins).

The invention is further described by reference to the following examples, which are provided for illustration only. The invention is not limited to the examples, but rather includes all variations that are evident from the teachings provided herein. All publicly available documents referenced herein, including but not limited to U.S. patents, are specifically incorporated by reference.

### EXAMPLES

### Example 1

The purpose of this example was to describe and analyze microarray data regarding HSS1.

Methods:

Cell culture: A172 glioma cell lines (ATCC, Manassas, Va., USA) were cultured in DMEM supplemented with 10% FBS (Life technologies, Grand Island, N.Y., USA). The human U87 glioma cell line (ATCC HTB-14) was maintained in alpha-MEM (ATCC, Manassas, Va., USA) supplemented with 10% fetal bovine serum (FBS). HUVECs (LONZA, Allendale, N.J., USA) were maintained in Endothelial Cell Growth Medium (EGM) (LONZA, Allendale, N.J., USA).

Stable Transfection:

The glioblastoma-derived A172 and U87 cell lines were stably transfected with hHSS1 as described in Junes-Gill et al., J. Neurooncol., 102(2):197-211 (2011). Stable clones were maintained with 500 ug ml⁻¹ of G-418 (Invitrogen, Carlsbad, Calif., USA) added to the cultures. The pcDNA3.1 construct used to stably express hHSS1 had a 6-His tag in-frame fused at the C-terminal of the hHSS1 gene.

Transcript Expression Profiling Using Microarray:

GeneChip Human Gene 1.0 ST Array (Affymetrix, Santa Clara, Calif., USA) was used to obtain transcript expression profiles in wild type (non-transfected), mock stable-transfected (pcDNA3.1 empty vector) and hHSS1-stable-transfected (pcDNA3.1-hHSS1) U87 and A172 cells. U87 cells (4×10⁵) were cultured in duplicate in 10 cm plates and incubated at 37° C., 5% CO₂. After 5 days, cells were harvested by trypsinization and viability determined by trypan blue exclusion. A172 cells (2×10⁵) were plated in triplicate in 10 cm plates and after 4 days the cells were harvested and counted. The expression profile of one clone of U87 cells and two clones of A172 cells (C#7 and C#8) expressing hHSS1 was evaluated. Expression of hHSS1 mRNA on stable clones was confirmed using qRT-PCR prior to microarray analysis. Total RNA was isolated using the RNeasy minikit (Qiagen, Valencia, Calif., USA). During the RNA purification process samples were treated with DNAse on the column before washing with buffer RPE. RNA characterization and chip analysis was carried out at the Functional Genomics Core of the City of Hope (Duarte, Calif., USA) and at the Core Facility of Children's Hospital Los Angeles (Los Angeles, Calif., USA). Technical replicates of U87 RNA samples were evaluated in triplicates and A172 cells were evaluated in biological triplicates. Expression values were determined using dChip (Jul. 9, 2009 build) or Partek software (St. Louis, Mo., USA).

Network and Pathways Analysis:

Ingenuity Pathway Analysis (IPA, Ingenuity® Systems, http://www.ingenuity.com, Redwood City, Calif., USA) was done using differentially expressed genes (DEGs) with P<0.001 with at least a 1.3 (A172 cells) and 1.5 (U87 cells) fold-change between hHSS1 expressing cells and control. For Ingenuity® iReport analysis (Ingenuity® Systems, http://www.ingenuity.com, Redwood City, Calif., USA), gene expression was considered significant at P<0.05 and a fold change cutoff of 2 (U87 cells) and 1.5 (A172 cells) were deemed significant. A lower cutoff was chosen for A172 cells because of the small number of DEGs. The scores generated by the network and pathway analysis are derived from a P-value and indicates the likelihood of the focus gene connectivity to be due to random chance. A score of 2 indicates that there is a 1 in 100 chance that the focus genes are together in a network due to random chance. Therefore, scores of 2 or higher have at least a 99% confidence of not being generated by random chance alone.

qRT-PCR:

Validation of DEGs from the microarray analysis was done by quantitative RT-PCR. cDNA synthesis was performed by reverse transcription of total RNA using Transcriptor First Strand cDNA Synthesis Kit (Roche, Indianapolis, Ind., USA). qRT-PCR was performed using gene-specific primers and hydrolysis probes (Biosearch Technologies, Petaluma, Calif., USA) and LightCycler 480 Probes Master Kit reagents (Roche, Indianapolis, Ind., USA). All reactions were performed in triplicate, using a total of 18 μl/well with primer concentration of 100 nM, in a LightCycler 480 System (Roche, Indianapolis, Ind., USA). Five different target genes were selected for each cell line. Each target was normalized to RPL32 housekeeping gene. Relative expression was calculated using LightCycler 480 Software 1.5 version (Roche, Indianapolis, Ind., USA). Fold-change was determined by the ratio between cells overexpressing hHSS1/cells overexpressing empty vector, and represented by fold-change if >1 and −1/fold-change if <1. Data were represented as mean values of biological triplicates (A172) and technical triplicates (U87).

Cell Cycle Analysis:

Exponentially growing U87 cells at growth curve day 4 and A172 cells at growth curve day 5 (Junes-Gill et al., J. Neurooncol., 102(2):197-211 (2011)) were harvested by trypsinization and stained with 50 μg/ml propidium iodide, 100 μg/mL RNAase DNase-free (Roche, Indianapolis, Ind., USA). DNA content and cell cycle distribution were analyzed by FACS (Beckman Counter, EPICS-XL, Fullerton, Calif., USA). Two independent experiments were performed.

Transwell Migration Assay:

BD BioCoat transwell chambers (BD Biosciences, Bedford, Mass., USA) with 8-μM pore size PET membrane inserts for 24-well plates were used according to the manufacturer instructions. Briefly, 5×10⁴ cells in serum free medium (DMEM or EMEM) were plated in the upper well of the transwell chambers, whereas medium supplemented with 10% FBS was added to the lower chamber as the chemoattractant. Following a 22 h incubation, the cells on the upper side of the inserts were removed using a cotton swab. The inserts were fixed in cold methanol and stained with hematoxylin and eosin (H&E, Sigma-Aldrich, St. Louis, Mo., USA). The number of migrated cells attached to the other side of the insert was counted from 9 random fields using a BX41 Olympus microscope (Center Valley, Pa., USA) equipped with 20× objective lens. Pictures were taken at a magnification of 200× using a DP73 camera (Olympus, Center Valley, Pa., USA) mounted on the microscope. Two independent experiments were done in duplicates. We performed a co-culture assay to verify a glioblastoma cell-induced migration of HUVEC cells. Briefly, U87 or A172 cells (2.5×10⁵) were seeded in the outer chamber of a 24-well plate with DMEM or EMEM supplemented with 2% FBS. Cells were allowed to adhere for 8 h at 37° C., 5% CO₂. After that, media was changed to serum-free media containing 0.1% BSA and incubated overnight at 37° C., 5% CO₂ for conditioned media production. Next day, 2.5×10⁴ HUVEC cells (1:10 ratio of glioblastoma cells) in serum-free media containing 0.1% BSA were seeded in the upper chamber. After 24 h, migrated cells from 21 fields were counted. Pictures were taken at a magnification of 200×. Two independent experiments were performed in duplicates.

Transwell Invasion Assay:

Invasion assays were performed using BD BioCoat Matrigel Invasion Chambers (BD Biosciences, San Jose, Calif., USA) according to the manufacturer instructions. Briefly, A172 or U87 (5×10⁴) cells in serum free medium (DMEM or EMEM) were plated in the upper well of the transwell chambers, whereas medium containing 10% FBS was placed into the lower chamber. The cells were allowed to invade thought the matrix for 24 h. After that, the cells growing on matrigel in the upper chamber were removed using a cotton swab. The inserts were fixed in cold methanol and stained with H&E. The number of invaded cells attached to the other side of the insert was counted from 9 random fields. Pictures were taken at a magnification of 200×. Two independent experiments were done in duplicates. Co-culture assay to verify a glioblastoma cell-induced invasion of HUVEC cells was performed. This experiment was done using the same conditions as mentioned above for the HUVEC co-culture migration assay, with the exception that inserts coated with matrigel were used. Two independent experiments were done in duplicate.

Angiogenesis Assay:

The angiogenesis in vitro assay was conducted in 96-well plates coated with 50 ul of ECMatrix™ (Millipore, Billerica, Mass., USA) following the manufacturer's instructions. HUVEC cells (2.5×10⁴ cells/well) were treated with purified hHSS1-his or vehicle control (PBS 1×) in EGM (LONZA, Allendale, N.J., USA) containing 1.2-1.5% FBS. Briefly, cells were pre-treated with 500 nM and 200 nM of hHSS1-his or vehicle control for 3 h at 37° C., 5% CO₂. Vehicle control was diluted following the protein dilution scheme. HUVECs were then plated onto matrigel-coated plates and incubated at 37° C., 5% CO₂ for 8 h to allow tube formation. After that, cells were stained with 0.5% crystal violet diluted in 50% ethanol and 5% formaldehyde and tube formation was evaluated. Two independent experiments were done in duplicate.

TCGA Database Analysis:

428 glioblastoma (GBM) samples were selected from the TCGA database that had both level 3 UNC Agilent G4502A microarray gene expression data and corresponding clinical information. A list of 12 genes was prospectively selected to correlate with hHSS1 gene expression. These genes were: ADAMTS1, APLN, BRCA1, BRCA2, CDKN2A, COL18A1 (endostatin), EGFR, JAM2, MMP9, RAD51, STATS, and THBS1. hHSS1 expression was compared with the selected genes using pairwise Pearson correlations, with r values ≧0.128 being considered significant. High and low hHSS1 expression (Log 2-transformed) was subdivided by the median expression level of the GBM cohort, and mean gene expression levels between high and low hHSS1 expression cohorts for each of the 12 genes was compared by the two-tailed Student's t-test. Differences were considered statistically significant when P<0.01.

Statistical Analysis:

Differences among groups in the cell cycle analysis were determined by one way ANOVA with Tukey's test for pairwise post-hoc comparisons. Differences were considered statistically significant when P<0.05. For the migration and invasion assays, two-tailed Student's t-test was performed to establish the statistical significance of differences between control cells and hHSS1-expressing cells. Differences were considered statistically significant when P<0.01.

Results

Overview of Microarray Analysis:

Exponentially growing A172 and U87 cells were harvested after 4 and 5 days, respectively. hHSS1-expressing cells and control cells were at confluence 40-80% when harvested. Trypan blue analysis of the number of viable cells showed a significant anti-proliferative effect in both cell lines expressing hHSS1 as compared to the control cells (A172/U87 wild-type and A172/U87-pcDNA3.1 empty vector).

Total RNA was analyzed on Affymetrix GeneChip Human Gene 1.0 ST Array which contains 28,869 genes represented by approximately 26 probes spread across the full length of the gene. These genes, along with their fold-change values, served as input to Ingenuity® iReport or IPA (Ingenuity® Systems, http://www.ingenuity.com). Canonical pathways are shown as depicted by Ingenuity® iReport or IPA. A right-tailed Fisher's exact test was used to identify over-represented functions/canonical pathways. The P-values derived through these analyses were based on: (1) total number of functions/canonical pathways eligible molecules that participate in that annotation; (2) total number of knowledge base molecules known to be associated with that function; (3) total number of functions/canonical pathways eligible molecules, and (4) total number of genes in the reference set.

Up-regulated and down-regulated genes in hHSS1-overexpressing A172 and U87 cells: With a cutoff value of a 2 fold change (FC), expression of 1,034 genes was significantly altered when hHSS1 was overexpressed in U87 cells. See Tables 1 and 2, below.

TABLE 1
21 most up-regulated genes following
hHSS1 overexpression in U87cells

| Symbol | Gene name | FC* |
|---|---|---|
| IL13RA2 | Interleukin 13 Receptor, Alpha 2 | 112.836 |
| CT45A5 | Cancer/testis Antigen Family 45, | 37.258 |
| | Member A5 | |
| ATP6V0D2 | Atpase, H+ Transporting, Lysosomal | 17.409 |
| | 38 kda, V0 Subunit D2 | |
| C3AR1 | Complement Component 3a Receptor 1 | 13.828 |
| IL1RN | Interleukin 1 Receptor Antagonist | 12.769 |
| PNLIPRP3 | Pancreatic Lipase-related Protein 3 | 11.422 |
| LOC654433 | Hypothetical Loc654433 | 11.361 |
| LOC151760 | Hypothetical Loc151760 | 10.637 |
| FAM198B | Family with Sequence Similarity 198, | 8.365 |
| | Member B | |
| GDF15 | Growth Differentiation Factor 15 | 8.017 |
| ANKRD1 | Ankyrin Repeat Domain 1 (Cardiac Muscle) | 7.661 |
| FBXO32 | F-box Protein 32 | 7.469 |
| RSPO3 | R-spondin 3 Homolog (Xenopus Laevis) | 7.223 |
| NR0B1 | Nuclear Receptor Subfamily 0, Group B, | 6.862 |
| | Member 1 | |
| IL1A | Interleukin 1, Alpha | 6.842 |
| GCNT3 | Glucosaminyl (N-acetyl) Transferase 3, | 6.809 |
| | Mucin Type | |
| GABRA2 | Gamma-aminobutyric Acid (Gaba) a | 6.791 |
| | Receptor, Alpha 2 | |
| NCAM2 | Neural Cell Adhesion Molecule 2 | 6.704 |
| ANO3 | Anoctamin 3 | 6.597 |
| ADAMTS5 | Adam Metallopeptidase with Thrombospondin | 6.263 |
| | Type 1 Motif, 5 | |
| CD55 | Cd55 Molecule, Decay Accelerating Factor | 6.159 |
| | for Complement (Cromer Blood Group) | |

*FC represents fold change at q ≦ 0.05 of a gene following hHSS1 modulation compared to cells stably transfected with vector control.

TABLE 2
37 most down-regulated genes following
h HSS1 overexpression in U87 cells

| Symbol | Gene name | FC* |
|---|---|---|
| DHCR24 | 24-dehydrocholesterol Reductase | −6.046 |
| FOS | Fbj Murine Osteosarcoma Viral | −6.103 |
| | Oncogene Homolog | |
| COL1A1 | Collagen, Type I, Alpha 1 | −6.132 |
| PDK3 | Pyruvate Dehydrogenase Kinase, | −6.236 |
| | Isozyme 3 | |
| PGF | Placental Growth Factor | −6.268 |
| CASC5 | Cancer Susceptibility Candidate 5 | −6.276 |
| KIF11 | Kinesin Family Member 11 | −6.342 |
| ERCC6L | Excision Repair Cross-complementing | −6.36 |
| | Rodent Repair Deficiency, | |
| | Complementation Group 6-like | |
| KIF15 | Kinesin Family Member 15 | −6.494 |
| SPC25 | Spc25, Ndc80 Kinetochore Complex | −6.902 |
| | Component, Homolog (S. Cerevisiae) | |
| C7orf68 | Chromosome 7 Open Reading Frame 68 | −7.093 |
| IGFBP1 | Insulin-like Growth Factor Binding | −7.129 |
| | Protein 1 | |
| FAM70A | Family with Sequence Similarity 70, | −7.265 |
| | Member A | |
| ESCO2 | Establishment of Cohesion 1 Homolog 2 | −7.283 |
| | (S. Cerevisiae) | |
| PTPRF | Protein Tyrosine Phosphatase, | −7.283 |
| | Receptor Type, F | |
| GPR155 | G Protein-coupled Receptor 155 | −7.323 |
| HIST1H2BM | Histone Cluster 1, H2bm | −7.326 |
| NID1 | Nidogen 1 | −7.326 |
| MKI67 | Antigen Identified by Monoclonal | −7.88 |
| | Antibody Ki-67 | |
| ELMO1 | Engulfment and Cell Motility 1 | −7.918 |
| DOK5 | Docking Protein 5 | −7.943 |
| FAM111B | Family with Sequence Similarity 111, | −7.975 |
| | Member B | |
| RRM2 | Ribonucleotide Reductase M2 | −8.078 |
| MYBL2 | V-myb Myeloblastosis Viral Oncogene | −8.361 |
| | Homolog (Avian)-like 2 | |
| IGFBP3 | Insulin-like Growth Factor Binding | −8.394 |
| | Protein 3 | |
| SLFN11 | Schlafen Family Member 11 | −8.461 |
| C4orf49 | Chromosome 4 Open Reading Frame 49 | −8.636 |
| FAM115C | Family with Sequence Similarity 115, | −10.234 |
| | Member C | |
| ACPP | Acid Phosphatase, Prostate | −10.234 |
| APLN | Apelin | −10.699 |
| GLB1L2 | Galactosidase, Beta 1-like 2 | −10.894 |
| TIMP3 | Timp Metallopeptidase Inhibitor 3 | −10.898 |
| MT1M | Metallothionein 1 m | −11.858 |
| BEND5 | Ben Domain Containing 5 | −12.104 |
| TXNIP | Thioredoxin Interacting Protein | −12.625 |
| HIST1H1A | Histone Cluster 1, H1a | −15.458 |
| THBS1 | Thrombospondin 1 | −18.526 |

*FC represents fold change at q ≦ 0.05 of a gene following hHSS1 modulation compared to cells stably transfected with vector control.

The molecules JUN, CDK1, VEGFA and FOS showed the highest connectivity ranking. The most down and up-regulated genes were functionally heterogeneous, among them were transcriptional regulators (ANKRD1, MYBL2), growth factors (GDF15, PGF) enzymes (SLFN11, DHCR24, FBXO32, GCNT3), transporters (ATP6V0D2), phosphatases (ACPP, PTPRF), peptidases (ADAMT55), cytokines (IL1RN, IL1A), kinases (PDK3, RSPO3), G-protein coupled receptors (GPR155, C3AR1) and transmembrane receptors (IL13RA2). There were many transcripts represented that did not have any known protein subcellular localization (CT45A5, PNLIPRP3, LOC654433, LOC151760, ANO3, MT1M, GLB1L2, FAM115C, C4orf49, FAM111B, FAM70A) (Tables 1 and 2).

The most up-regulated genes in U87 cells were interleukins and receptors (IL1A. IL13RA2, and IL1RN), CT45A5 from the cancer/testis (CT) family of antigens, and the cytoplasmic transporter ATP6V0D2 (Table 1). The most down-regulated genes were thrombospondin 1 (THBS1) and histone cluster 1 (HIST1H1A). Among the most down-regulated genes in U87 is apelin (APLN), a ligand for the angiotensin-like 1 (APJ) receptor (O'Dowd et al., “A human gene that shows identity with the gene encoding the angiotensin receptor is located on chromosome 11,” Gene., 136(1-2):355-360 (1993); and Tatemoto et al., “Isolation and characterization of a novel endogenous peptide ligand for the human APJ receptor,” Biochem. Biophys. Res. Commun., 251(2):471-476 (1998)) and a novel factor involved in angiogenesis (Table 2).

84 differentially expressed genes were identified in A172 cells due to hHSS1 overexpression, when a lower FC cutoff of 1.5 was used (Tables 3 and 4, below).

TABLE 3
Total list of most up-regulated genes following
h HSS1 overexpression in A172 cells

| Symbol | Gene name | FC* |
|---|---|---|
| C19orf63 | Chromosome 19 Open Reading Frame 63 | 11.881 |
| ZNF22 | Zinc Finger Protein 22 (Kox 15) | 4.012 |
| KRT81 | Keratin 81 | 3.93 |
| AADAC | Arylacetamide Deacetylase (Esterase) | 3.317 |
| AMTN | Amelotin | 3.018 |
| JAM2 | Junctional Adhesion Molecule 2 | 2.66 |
| FAM133A | Family with Sequence Similarity 133, Member A | 2.606 |
| EDIL3 | Egf-like Repeats and Discoidin I-like Domains 3 | 2.524 |
| C2orf15 | Chromosome 2 Open Reading Frame 15 | 2.299 |
| CLDN1 | Claudin 1 | 2.239 |
| BICC1 | Bicaudal C Homolog 1 (Drosophila) | 2.092 |
| IL2RG | Interleukin 2 Receptor, Gamma | 1.895 |
| SYTL5 | Synaptotagmin-like 5 | 1.887 |
| KAL1 | Kallmann Syndrome 1 Sequence | 1.875 |
| CDH10 | Cadherin 10, Type 2 (T2-cadherin) | 1.861 |
| SLC25A27 | Solute Carrier Family 25, Member 27 | 1.839 |
| TAF4B | Taf4b Rna Polymerase Ii, Tata Box Binding | 1.837 |
| | Protein (Tbp)-associated Factor, 105 kda | |
| ACTA2 | Actin, Alpha 2, Smooth Muscle, Aorta | 1.821 |
| NAP1L3 | Nucleosome Assembly Protein 1-like 3 | 1.795 |
| PLEKHA1 | Pleckstrin Homology Domain Containing, | 1.757 |
| | Family a (Phosphoinositide Binding Specific) | |
| | Member 1 | |
| IL18 | Interleukin 18 (Interferon-gamma-inducing | 1.708 |
| | Factor) | |
| KCTD16 | Potassium Channel Tetramerisation Domain | 1.689 |
| | Containing 16 | |
| ZNF571 | Zinc Finger Protein 571 | 1.653 |
| INPP5A | Inositol Polyphosphate-5-phosphatase, 40 kda | 1.643 |
| ZMAT1 | Zinc Finger, Matrin-type 1 | 1.642 |
| DOCK1 | Dedicator of Cytokinesis 1 | 1.617 |
| TSGA10 | Testis Specific, 10 | 1.598 |
| CADM1 | Cell Adhesion Molecule 1 | 1.592 |
| ECHS1 | Enoyl Coa Hydratase, Short Chain, I, | 1.584 |
| | Mitochondrial | |
| ENTPD1 | Ectonucleoside Triphosphate | 1.573 |
| | Diphosphohydrolase 1 | |
| ZRANB1 | Zinc Finger, Ran-binding Domain Containing 1 | 1.567 |
| PTPRE | Protein Tyrosine Phosphatase, Receptor Type, E | 1.548 |
| TP53INP1 | Tumor Protein P53 Inducible Nuclear Protein 1 | 1.543 |
| DUSP10 | Dual Specificity Phosphatase 10 | 1.543 |
| TM2D1 | Tm2 Domain Containing 1 | 1.527 |
| ZMAT3 | Zinc Finger, Matrin-type 3 | 1.522 |
| LTBP2 | Latent Transforming Growth Factor Beta | 1.516 |
| | Binding Protein 2 | |

*FC represents fold change at q ≦ 0.05 of a gene following hHSS1 modulation compared to cells stably transfected with vector control.

TABLE 4
Total list of most down-regulated genes following
h HSS1 overexpression in A172 cells

| Symbol | Gene name | FC* |
|---|---|---|
| MCM6 | Minichromosome Maintenance Complex | −1.501 |
| | Component 6 | |
| C6orf52 | Chromosome 6 Open Reading Frame 52 | −1.501 |
| FERMT3 | Fermitin Family Member 3 | −1.533 |
| SMC2 | Structural Maintenance of Chromosomes 2 | −1.546 |
| SRPX | Sushi-repeat Containing Protein, X-linked | −1.549 |
| SHCBP1 | Shc Sh2-domain Binding Protein 1 | −1.564 |
| GPSM2 | G-protein Signaling Modulator 2 | −1.564 |
| NES | Nestin | −1.565 |
| SYCP2 | Synaptonemal Complex Protein 2 | −1.575 |
| MCM10 | Minichromosome Maintenance Complex | −1.576 |
| | Component 10 | |
| EZH2 | Enhancer of Zeste Homolog 2 (Drosophila) | −1.58 |
| TMTC2 | Transmembrane and Tetratricopeptide Repeat | −1.594 |
| | Containing 2 | |
| FAM129A | Family with Sequence Similarity 129, Member A | −1.596 |
| TMEFF2 | Transmembrane Protein with Egf-like and Two | −1.604 |
| | Follistatin-like Domains 2 | |
| CTSL2 | Cathepsin L2 | −1.613 |
| ETV1 | Ets Variant 1 | −1.614 |
| SGOL2 | Shugoshin-like 2 (S. Pombe) | −1.62 |
| ERCC6L | Excision Repair Cross-complementing Rodent | −1.621 |
| | Repair Deficiency, Complementation Group | |
| | 6-like | |
| KRT15 | Keratin 15 | −1.641 |
| SDPR | Serum Deprivation Response | −1.656 |
| ACAT2 | Acetyl-coa Acetyltransferase 2 | −1.7 |
| BDKRB1 | Bradykinin Receptor B1 | −1.709 |
| CFI | Complement Factor 1 | −1.711 |
| GPD2 | Glycerol-3-phosphate Dehydrogenase 2 | −1.722 |
| | (Mitochondrial) | |
| TMOD1 | Tropomodulin 1 | −1.729 |
| FAM64A | Family with Sequence Similarity 64, Member A | −1.755 |
| ANO5 | Anoctamin 5 | −1.782 |
| LRRC15 | Leucine Rich Repeat Containing 15 | −1.812 |
| PAGE1 | P Antigen Family, Member 1 (Prostate | −1.822 |
| | Associated) | |
| XRCC2 | X-ray Repair Complementing Defective Repair in | −1.863 |
| | Chinese Hamster Cells 2 | |
| EMP2 | Epithelial Membrane Protein 2 | −1.868 |
| CD180 | Cd180 Molecule | −1.926 |
| ELOVL6 | Elovl Fatty Acid Elongase 6 | −1.931 |
| PLCXD3 | Phosphatidylinositol-specific Phospholipase C, | −1.938 |
| | X Domain Containing 3 | |
| C7orf69 | Chromosome 7 Open Reading Frame 69 | −1.941 |
| DMD | Dystrophin | −1.947 |
| MNS1 | Meiosis-specific Nuclear Structural 1 | −1.949 |
| FAM115C | Family with Sequence Similarity 115, Member C | −2.005 |
| TEK | Tek Tyrosine Kinase, Endothelial | −2.099 |
| CHRM3 | Cholinergic Receptor, Muscarinic 3 | −2.122 |
| RGS16 | Regulator of G-protein Signaling 16 | −2.144 |
| SULT1B1 | Sulfotransferase Family, Cytosolic, 1b, | −2.478 |
| | Member 1 | |
| ANKRD30B | Ankyrin Repeat Domain 30b | −2.592 |
| B3GALT1 | Udp-gal: betaglcnac Beta | −2.841 |
| | 1,3-galactosyltransferase, Polypeptide 1 | |
| XIRP2 | Xin Actin-binding Repeat Containing 2 | −3.387 |
| POTEB | Pote Ankyrin Domain Family, Member B | −6.162 |
| | (includes others) | |
| CCDC102B | Coiled-coil Domain Containing 102b | −11.348 |

*FC represents fold change at q ≦ 0.05 of a gene following hHSS1 modulation compared to cells stably transfected with vector control.

Thus, overexpression of hHSS1 had a larger effect in U87 as compared to A172 cells. KRT15 and MCM10 were the molecules with highest connectivity. Among the most up-regulated genes in A172 cells were zinc finger protein 22 (ZNF22), keratin 81 (KRT81), the enzyme arylacetamide deacetylase (AADAC), and the extracellular protein amelotin (AMTN) (Table 3). The most down-regulated were the coiled-coil domain containing 102b (CCDC102B) and the pote ankyrin domain family member B (POTEB) (Table 4).

Fifteen genes were concordantly altered in both U87 and A172 cell lines, 14 were down-regulated (JAM2, FAM115C, MNS1, ERCC6L, EMP2, EZH2, TMOD1, GPSM2, XRCC2, SGOL2, SMC2, FAM64A, MCM10, SHCBP1), and 1 was up-regulated (TAF4B). Two genes were altered in different direction with hHSS1 overexpression: the complement factor I (CFI) was up-regulated in U87 cells (FC: 2.9) while it was down-regulated in A172 cells (FC:−1.7). Likewise, tek tyrosine kinase (TEK) was up-regulated in U87 (FC: 2.2) but it was down-regulated (FC:−2.1) in A172 cells.

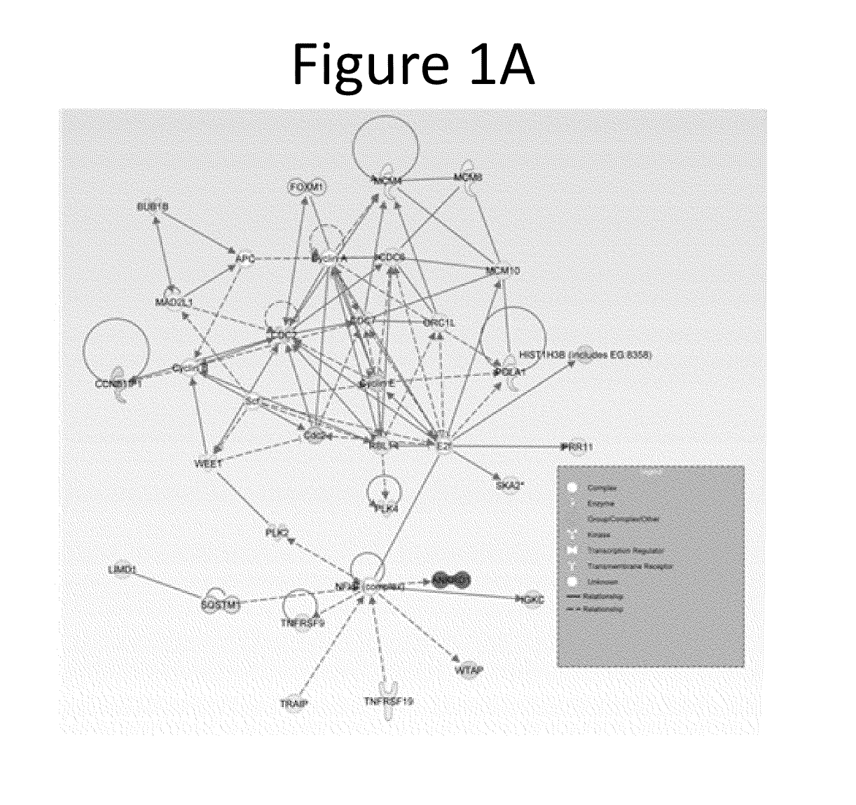

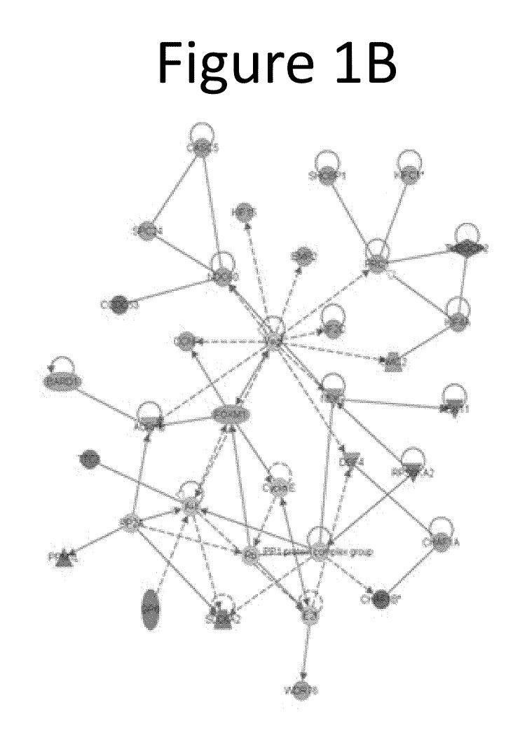

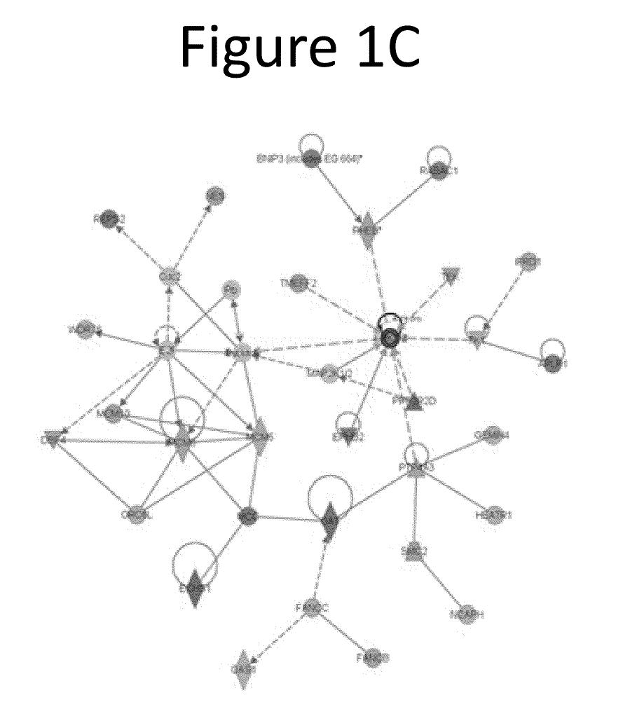

Network, pathway and functional analysis of genes influenced by hHSS1 overexpression in human U87 and A172 glioma cell lines: The interaction and functional importance of the signaling pathways involving genes significantly modulated by hHSS1 were evaluated. The list of differentially expressed genes analyzed by IPA revealed significant networks and interactions. FIG. 1 shows the top networks identified by IPA in both U87 and A172 cells. The highest significant network with 27 focus molecules and a significance score of 43 in the U87 cell dataset revealed genes related to the cell cycle, cell death, DNA replication, recombination and repair (FIG. 1A). There was a significant up-regulation of ANKRD1, a nuclear factor that has negative transcriptional activity in endothelial cells. Zou et al., “CARP, a cardiac ankyrin repeat protein, is downstream in the Nkx2-5 homeobox gene pathway,” Development, 124(4):793-804 (1997).

FIG. 1B shows the top network found in A172-hHSS1 clone #7. With a score of 48, the top network included molecules involved in cell cycle, cellular assembly and organization, DNA replication, recombination and repair. The highest significant network in A172-hHSS1 C#8 with a significance score of 50 revealed genes related to tissue morphology and cellular development (FIG. 1C).

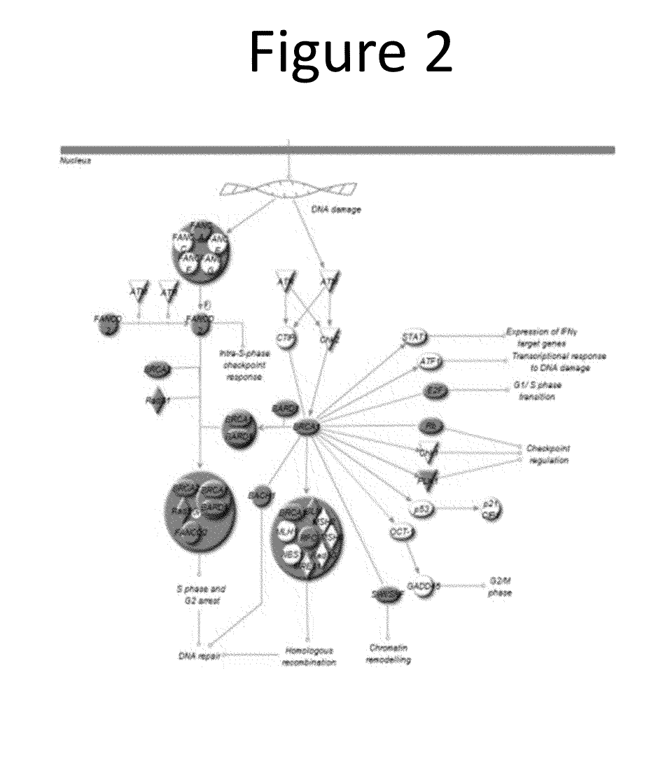

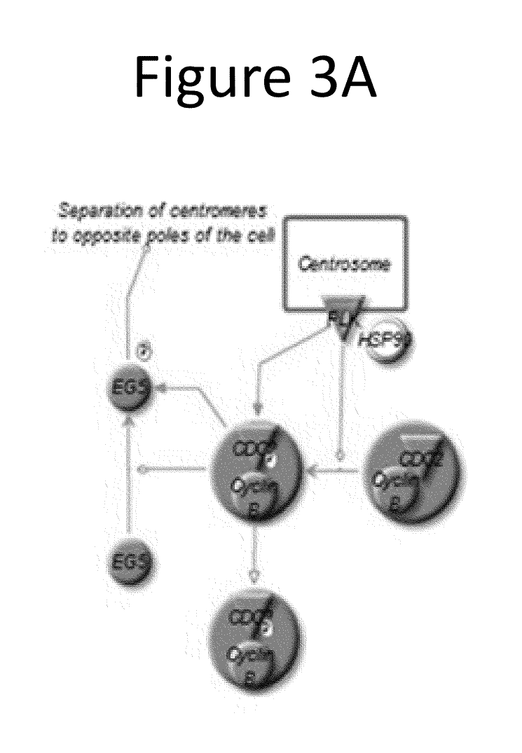

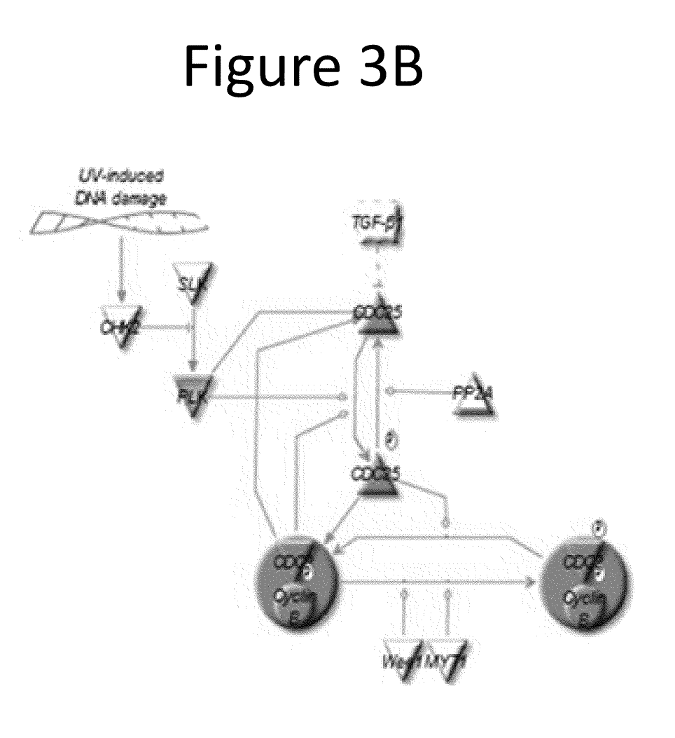

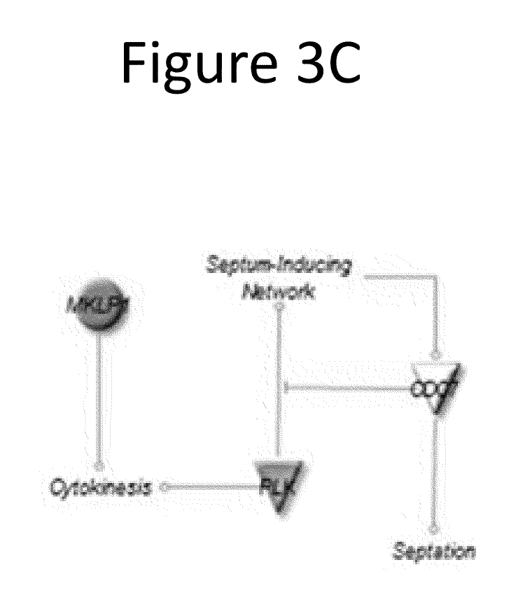

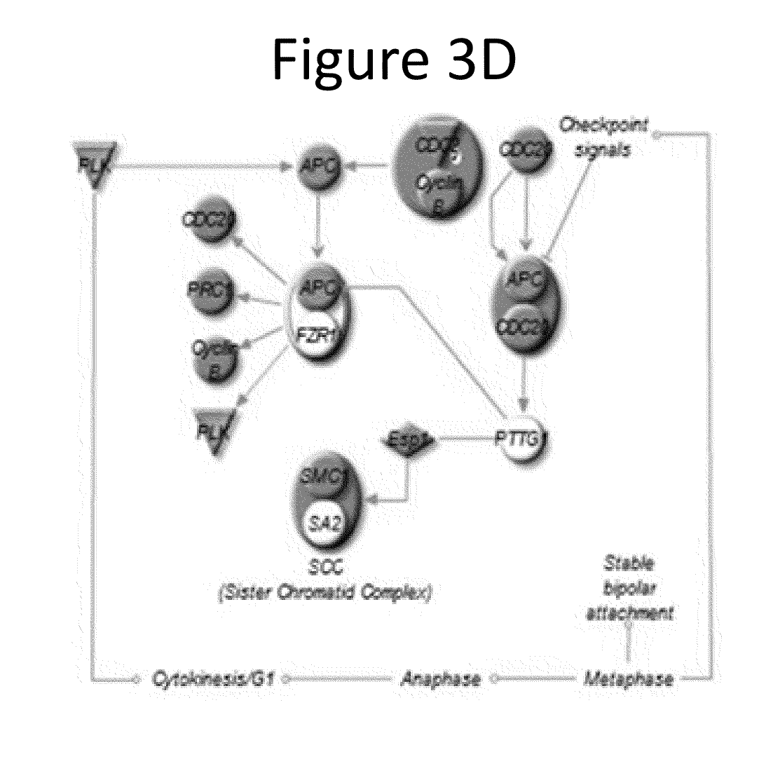

The pathway analysis of U87 cells strongly suggest that hHSS1 modulates genes related to the role of BRCA1 in DNA damage response (17 DEGs, P=1.70e⁻⁹), ATM signaling (13 DEGs, P=1.69e⁻⁶), and the mitotic roles of polo-like kinases pathway (14 DEGs, P=2.53e⁻⁶). The top most significant pathway showed that 17 differentially expressed genes in U87 cells were related to the DNA damage response involving members of the BRCA family (FIG. 2). hHSS1 down-regulated complexes of protein, namely BRCA1, BRCA2, Rad51, BARD and FANCD2 in U87 cells. These proteins are responsible for regulating the S and G2 phases of cell cycling. Genes involved in homologous recombination and chromatin remodeling were also down-regulated. The transcriptional regulator E2F5 responsible for the GUS phase transition was the only gene up-regulated in this pathway. The top 3 pathway in U87 cells regulated by hHSS1 was related to genes involved in the mitotic roles of polo-like kinases (FIG. 3), which included genes involved in centrosome separation and maturation (EG5, CDC2 and cyclin B), mitotic entry (CDC25, PLK, CDC2 and cyclin B) and metaphase and anaphase transition (APC, CDC20, PRC1, cyclin B. SMC1 and Esp1). Moreover, the functional analysis of differentially expressed genes in U87 cells, robustly suggested that hHSS1 affects the cell division process of chromosomes (57 DEGs, P=7.75e⁻²⁵), segregation of chromosomes (34 DEGs, P=4.49e⁻²³), mitosis (73 DEGs, P=2.33e⁻¹⁹), M phase (45 DEGs, P=1.53e⁻¹⁷), cell cycle progression (120 DEGs, P=2.86e⁻¹⁶), cell death of tumor cell lines (141 DEGs, P=1.80e⁻¹⁵) and proliferation of cells (235 DEGs, P=2.23e⁻¹⁵).

In A172 cells, the most significant pathways affected by hHSS1 overexpression were related to metabolism. Among them were butanoate and propanoate metabolism and the pathways related to valine, leucine and isoleucine degradation. The top most significant pathway was the butanoate metabolic pathway (A172-hHSS1 C#7: 5 DEGs, P=4.35e⁻⁵; A172-hHSS1 C#8: 4 DEGs, P=1.41e⁻⁴). Four genes were differentially expressed: AADAC and ECHS1 were up-regulated while ACAT2 and ELOVL6 were down-regulated. The most affected biological processes in A172 cells were cell-cell contact (A172-hHSS1 C#8: 5 DEGs, P=1.10e⁻⁴), growth of melanoma cell lines (A172-hHSS1 C#8: 3 DEGs, P=1.49e⁻³) and migration of embryonic cell lines (A172-hHSS1 C#8: 3 DEGs, P=2.25e⁻³). The biological process analysis was not determined for A172-hHSS1 C#7.

Validation of Microarray Data at the RNA Level:

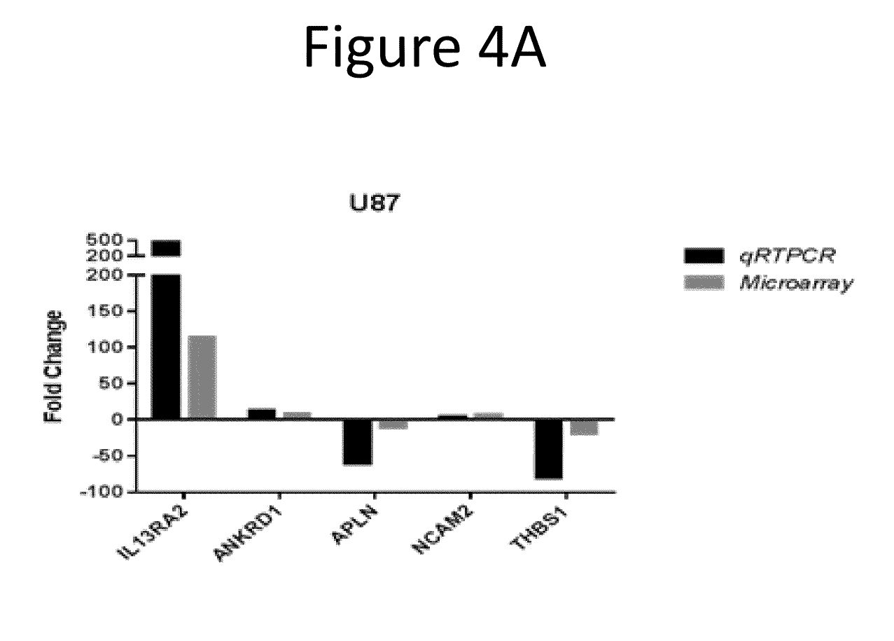

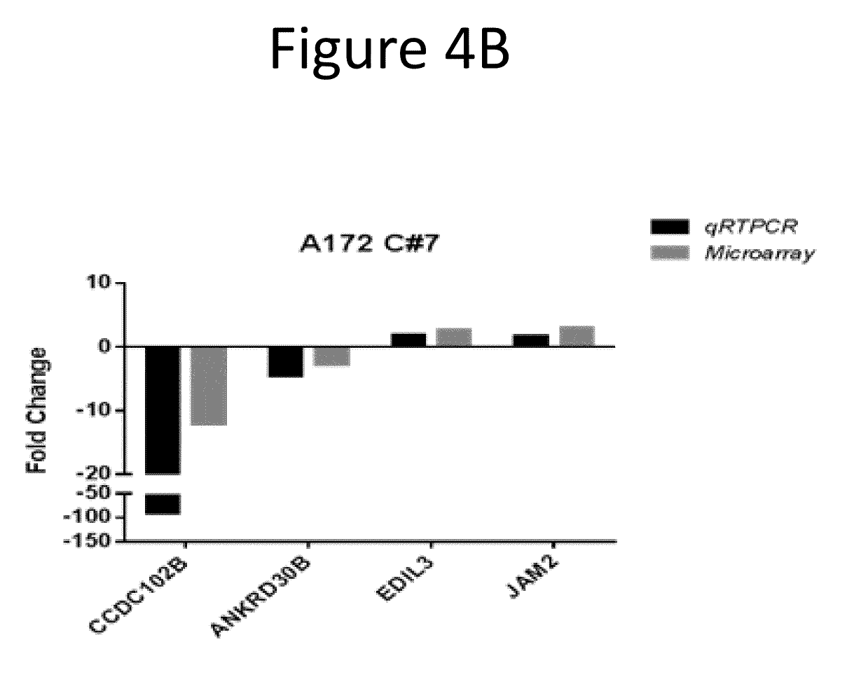

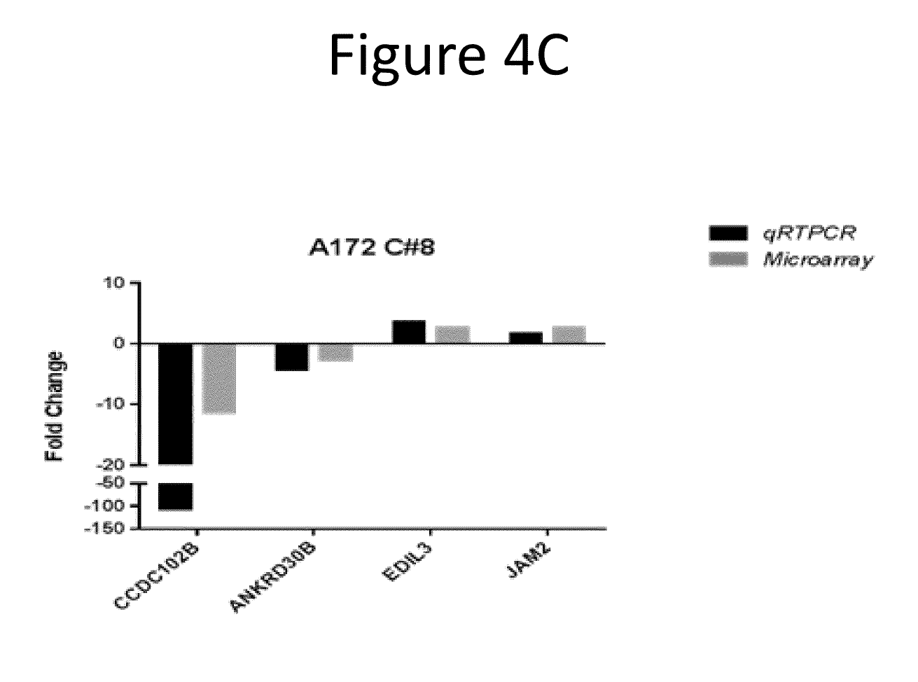

For validation of microarray data, a sub-set of differentially expressed genes were selected corresponding to the highest fold-change and particularly those which were involved with proliferation, adhesion, migration and invasion. Changes in gene expression were assessed using qRT-PCR for five different genes for each cell line: CCDC102B, XIRP2, ANKRD30B, EDIL3 and JAM2 for A172 cells evaluation; and the genes IL13RA2, ANKRD1, APLN, NCAM2 and THBS1 for U87 cells. From the genes selected for validation, only XIRP2 showed a discrepancy in gene expression between qRT-PCR and microarray analysis for both A172 C#7 and C#8 clones (FIG. 4).

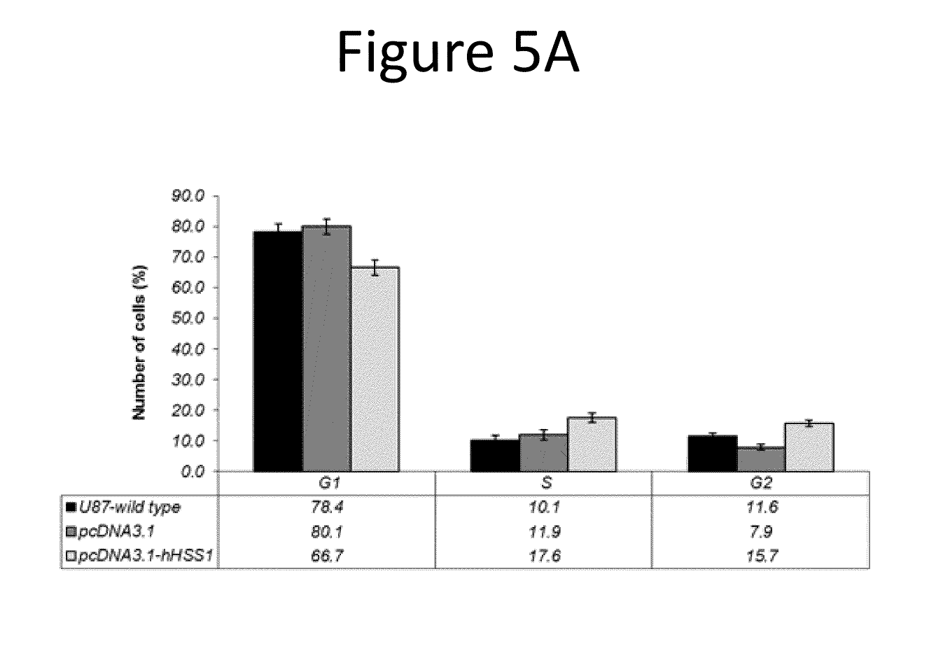

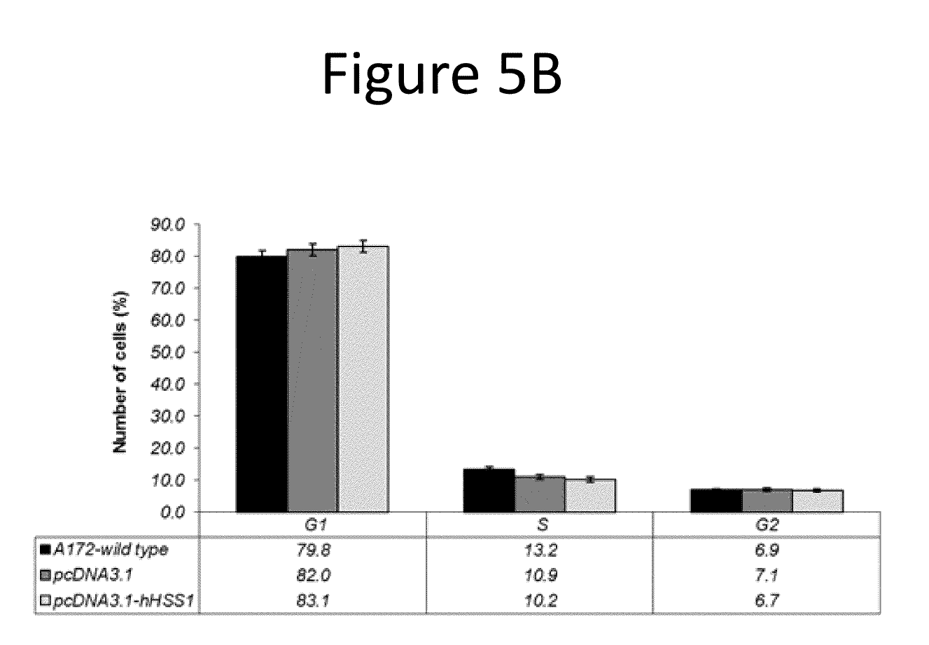

Effect of hHSS1 overexpression on cell cycle phases in human U87 and A172 glioma cell lines: Next, the cell cycle phases in U87 and A172 cells were evaluated to corroborate the microarray findings of differentially expressed genes in pathways related to cell cycle regulation. Previously it was shown that cell proliferation significantly decreased in cells overexpressing hHSS1 and observed a 5 and 10 hours delay in doubling time for U87 and A172, respectively (Junes-Gill (2011)). The cell cycle analysis of day 4 and 5 from U87 and A172 cells respectively, showed a significant decrease of cells in phases G0/G1, while a significant increase in cells was seen in S and G2/M phases in U87 cells overexpressing hHSS1 (FIG. 5). No difference in cell cycle distribution was observed for A172 cells, except for a significant decrease in S phase for A172-hHSS1 expressing cells compared with A172-wild type. Taken together, these results indicate that hHSS1 overexpression in A172 cells does not regulate a specific cell cycle phase but could prevent the overall progression of the cell cycle once it leads to a 10 hours delay in doubling time. This finding is consistent with the observed modulation of genes related to metabolic pathways.

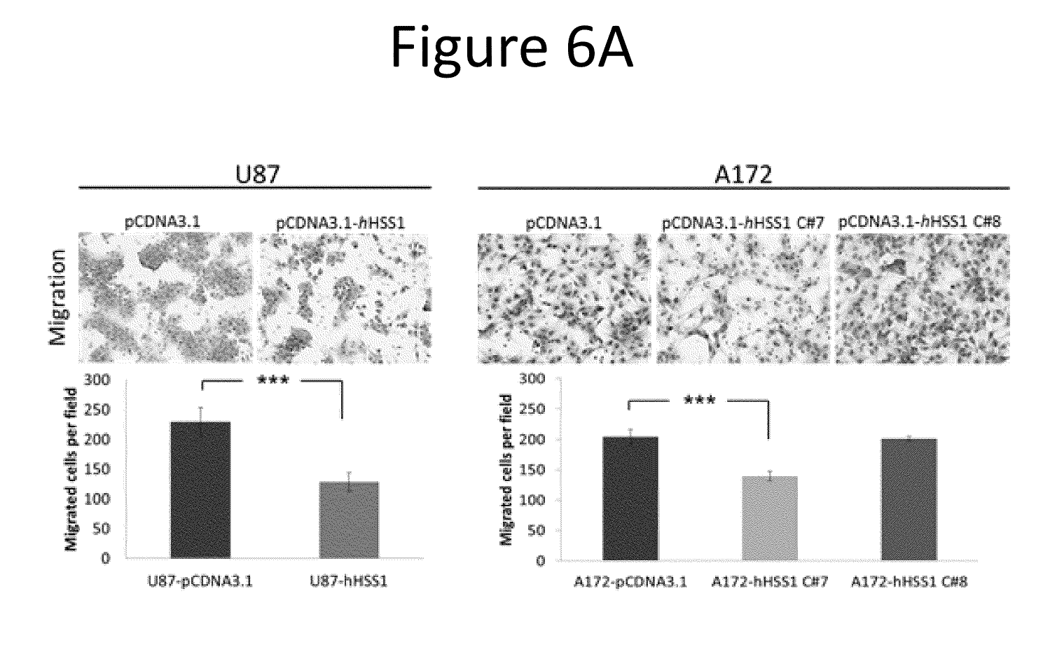

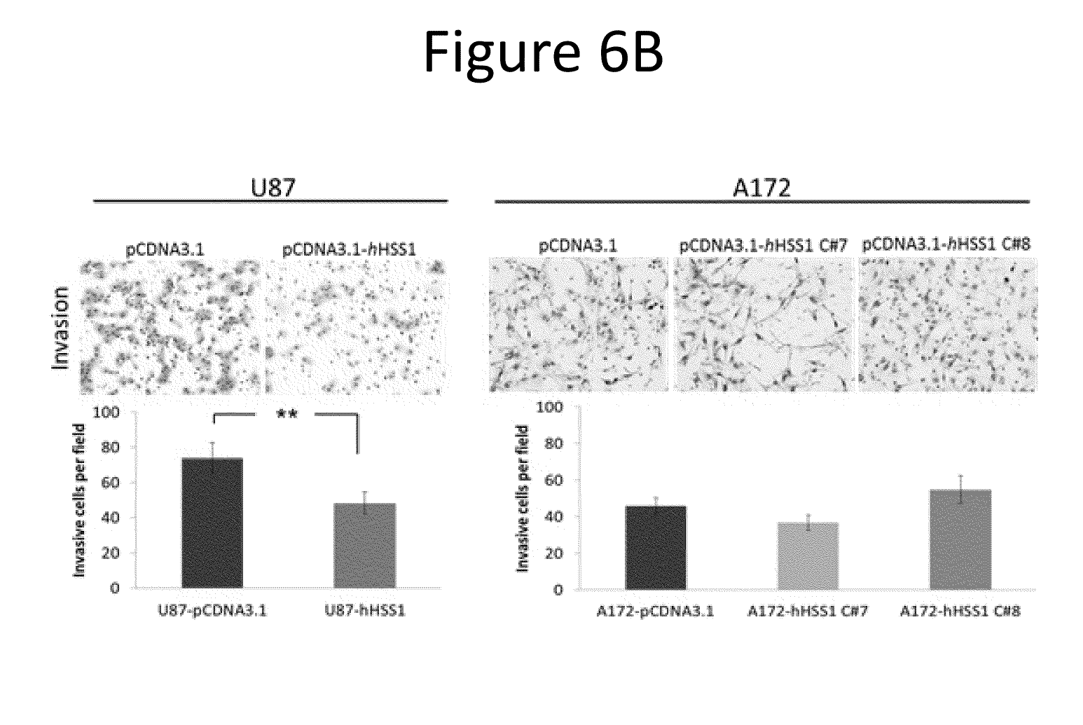

hHSS1 overexpression inhibits migration and invasion of human U87 and A172 glioma cell lines: One of the hallmarks of glioblastoma cells is that they infiltrate surrounding normal brain tissue and so lose constraints on cell migration. The microarray analysis described herein indicated that hHSS1 up or down regulated genes involved in cell migration, invasion and angiogenesis. To clarify an effect of hHSS1 on these key events involved in tumorigenesis, the modified Boyden chamber assay was used to study the migratory and invasive properties of U87 and A172 cells overexpressing hHSS1 (FIG. 6). U87 cells overexpressing hHSS1 significantly lost their ability to migrate and invade through a matrigel matrix, as compared to the U87-pcDNA.3.1 control cells. For A172 cells, C#7 but not C#8, showed a significant decrease in cell migration as compared with the control. Moreover, hHSS1 had no effect on A172 invasion, indicating that overexpression of hHSS1 does not have a consistent effect on the migratory and invasive properties of A172 cells. Taken together, the data demonstrate that overexpression of hHSS1 decreases the invasive properties of U87 cells but has no effect on A172 cells.

### Example 2

hHSS1 overexpression by human U87 and A172 glioma cell lines inhibited tumor-induced migration and invasion of HUVEC: The migration and invasion of endothelial cells through basement membranes are crucial steps in the development of new blood vessels. Stimulation of endothelial cells by tumor cells is known for establishing an endothelial phenotype consistent with the initial stages of angiogenesis. Khodarev et al., “Tumour-endothelium interactions in co-culture: coordinated changes of gene expression profiles and phenotypic properties of endothelial cells,” J. Cell Sci., 116(Pt 6):1013-1022 (2003).

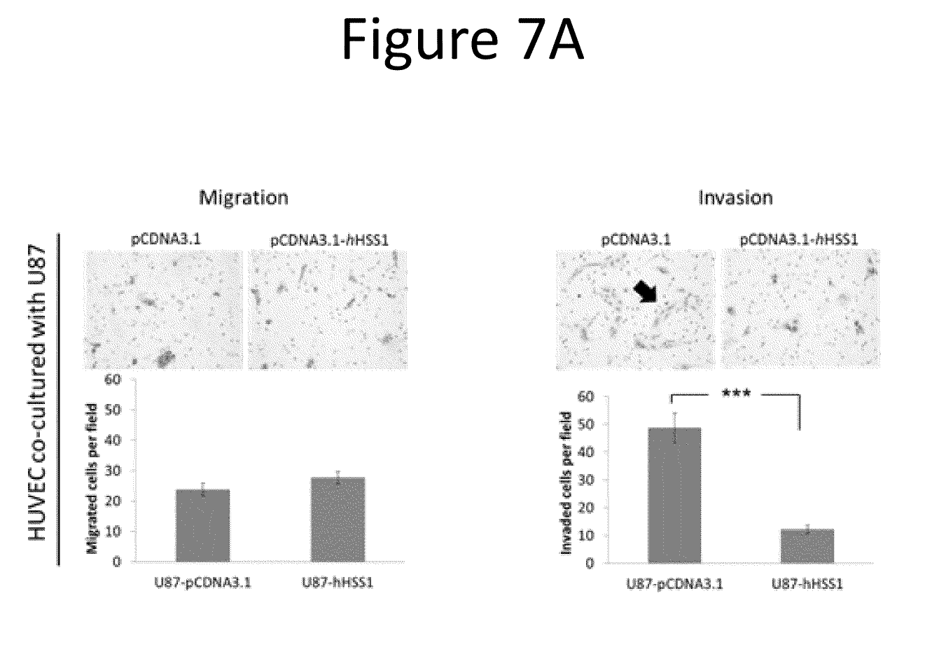

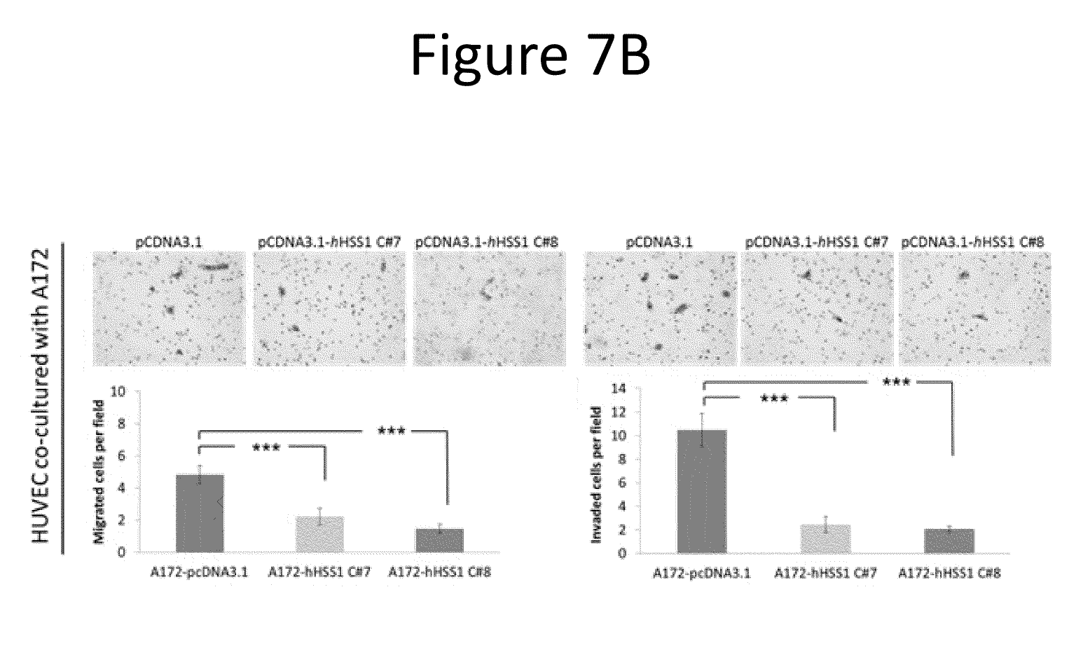

To determine if hHSS1 had an effect on angiogenesis, as suggested by the microarray analysis of Example 1, the ability of hHSS1 to impact these critical events in a co-culture assay was evaluated. Glioma cells overexpressing hHSS1 and HUVEC were co-cultured in transwell chambers, and the tumor-induced migration and invasion of HUVEC through matrigel was estimated (FIG. 7). At a 1:10 HUVEC:U87 ratio, there was a significant decrease in the invasion of HUVEC co-cultured with U87-hHSS1 cells as compared to HUVEC co-cultured with U87-pcDNA3.1 control. However, overexpression of hHSS1 did not affect the migration of HUVEC cells co-cultured with U87 cells. It was previously reported that U87 cells promote morphogenetic changes in HUVEC, including the formation of net-like structures resembling neo-vasculature (Khodarev 2003). Endothelial cells that invaded the matrix, in co-culture with U87-pcDNA3.1 control cells, appeared elongated with a narrower extended shape and aligned themselves to form net-like structures (FIG. 7A, black arrow). In contrast, HUVEC co-cultured with U87-hHSS1 had a rounded or ‘teardrop-like’ morphology, and did not align themselves to form net-like structures (FIG. 7A). HUVEC growing in co-culture with A172 C#7 and C#8 overexpressing hHSS1, displayed significant decrease in both migration and invasion ability when compared to HUVEC co-cultured with A172-pCDNA3.1 control cells (FIG. 7B).

These findings indicate that hHSS1 can impact angiogenesis, as it suppresses the tumor-induced HUVEC phenotype related to cell migration and invasion.

### Example 3

### Purified hHSS1 Protein Inhibits In Vitro Angiogenesis

The migration and invasion of endothelial cells are essential for the formation of new blood vessels during neo-angiogenesis, and consequently are critical events for tumor growth. Because ectopic overexpression of hHSS1 in glioma-derived cells strongly inhibited HUVEC cell migration and invasion, the effect of purified hHSS1 on the potential of HUVEC to form capillary-like structures was examined.

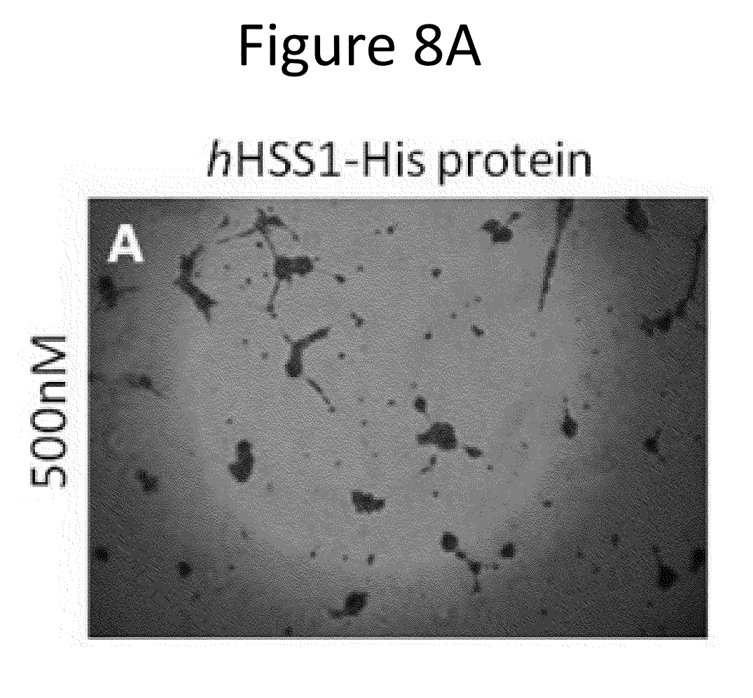

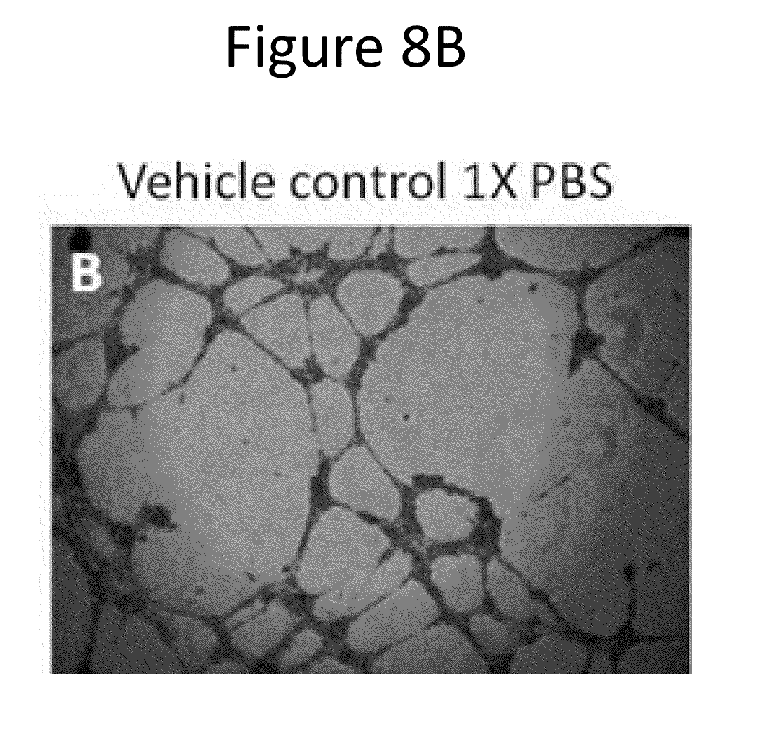

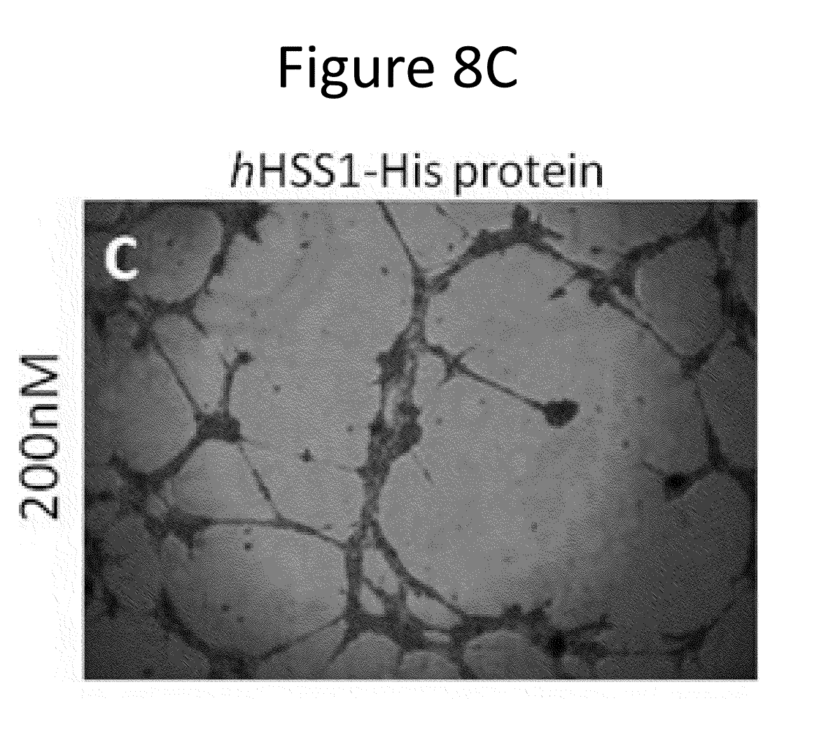

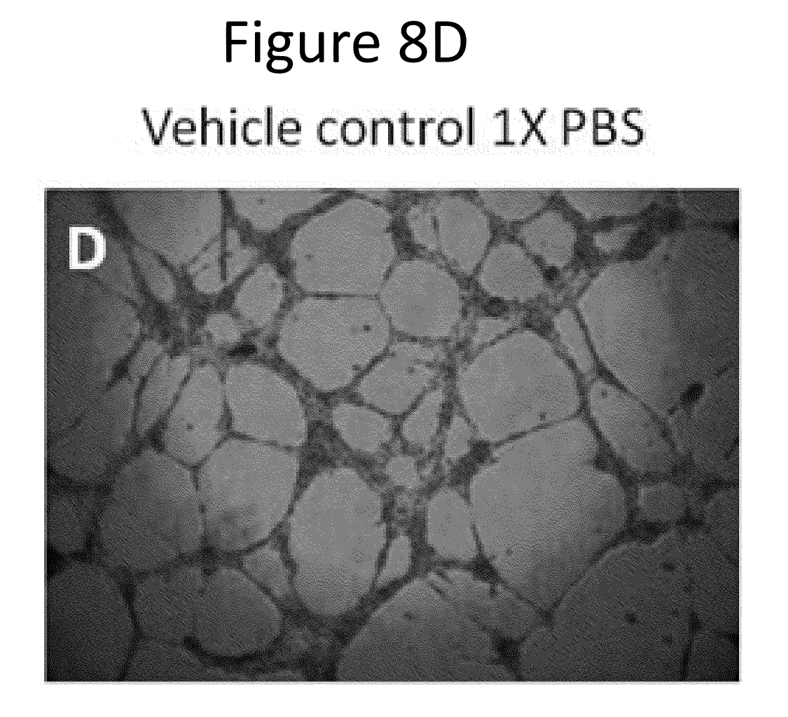

As shown in FIG. 8, HUVEC growing on matrigel treated with vehicle control formed complex network of tubes after 8 h, which was inhibited and disrupted in a concentration-dependent manner by treatment with 500 nM or 200 nM of purified hHSS1.

### Example 4

### hHSS1 Expression in GBM Samples from the TCGA Database

hHSS1 mRNA expression in 428 GBM samples from the TCGA database was compared to a list of 12 genes selected based on their involvement in GBM, invasion, migration, angiogenesis and significant pathways or networks identified from the U87/A172 cells overexpressing hHSS1.

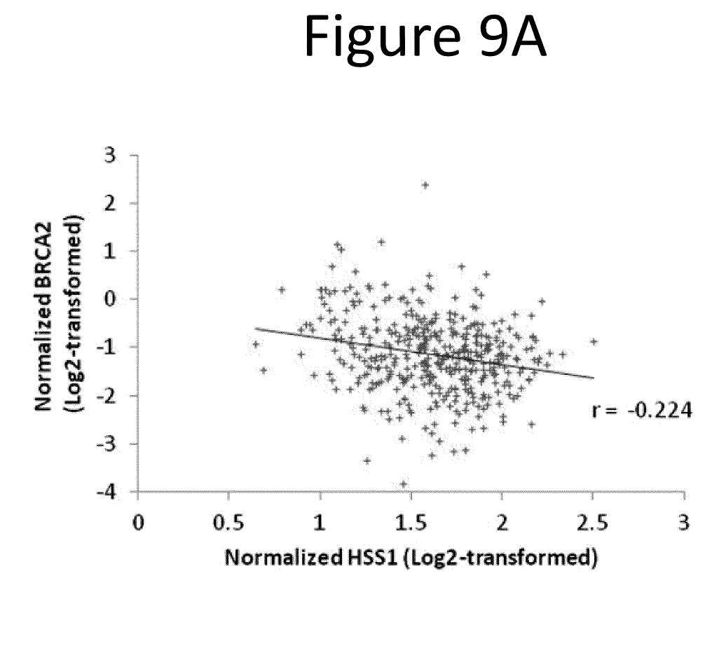

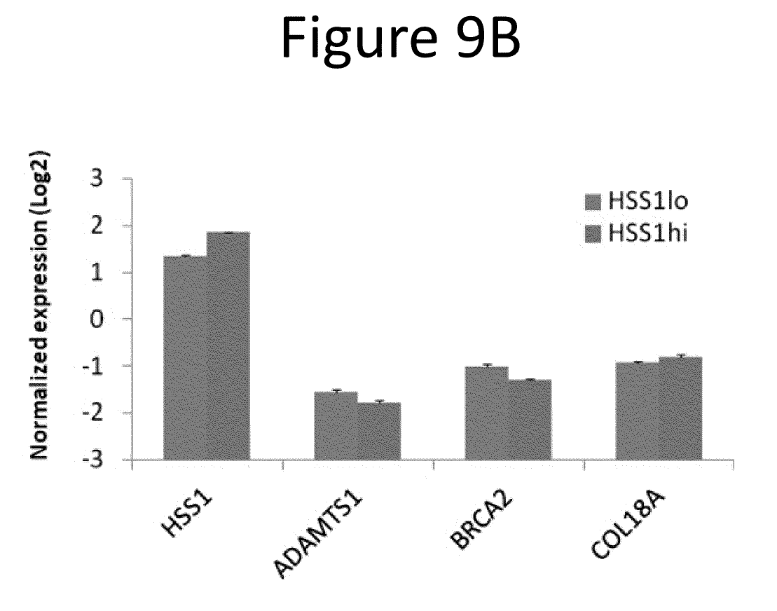

This analysis revealed a highly significant but weak inverse correlation with BRCA2 (r=−0.224, P<0.0005) (FIG. 9A). Moreover, statistically significant inverse correlation with ADAMTS1 (r=−0.132, P<0.01) and direct correlation with endostatin (r=0.141, P<0.005) were found (data not shown). The subdivision of the GBM cohort based on high and low hHSS1 expression showed that the levels of BRCA2 and ADAMTS1 expression on hHSS1-high expression group are significantly lower compared to hHSS1-low expression group (P<0.00006 and P<0.014, respectively) (FIG. 9B). Additionally, higher expression of endostatin was significantly found in hHSS1-high expression group compared to HSS1-low expression group (P<0.048).

The above examples are given to illustrate the present invention. It should be understood, however, that the spirit and scope of the invention is not to be limited to the specific conditions or details described in these examples. All publicly available documents referenced herein, including but not limited to U.S. patents, are specifically incorporated by reference.

What is claimed is:

1. A method for treating a non-brain cancer comprising administering to a subject in need a composition comprising at least one compound selected from the group consisting of:
(a) Hematopoietic Signal peptide-containing Secreted 1 (HSS1);
(b) Hematopoietic Signal peptide-containing Membrane domain-containing 1 (HSM1);
(c) a peptide having at least about 80% homology to HSS1, wherein the peptide exhibits anti-angiogenic activity;
(d) a peptide having at least about 80% homology to HSM1, wherein the peptide exhibits anti-angiogenic activity;
(e) a HSS1 fragment comprising at least 4 contiguous amino acids from the HSS1 amino acid sequence, wherein the fragment exhibits anti-angiogenic activity;
(f) a HSM1 fragment comprising at least 4 contiguous amino acids from the HSM1 amino acid sequence, wherein the fragment exhibits anti-angiogenic activity; and
(g) any combination thereof.

2. The method of claim 1, further comprising administering a conventional cancer therapy to the subject, wherein the conventional cancer therapy is surgery, radiation therapy, chemotherapy, or any combination thereof.

3. The method of claim 2, wherein the conventional cancer therapy is radiation therapy, and the composition radiosensitizes the cancer.

4. The method of claim 2, wherein the composition is administered before, during, or after the conventional cancer therapy.

5. The method of claim 1, wherein the cancer patient population to be treated is a carrier of BRCA1-2.

6. The method of claim 1, wherein the composition increases the survival of the subject.

7. The method of claim 1, wherein the subject has a tumor, and the composition results in slowing or inhibition of tumor growth, tumor progression, and/or tumor metastasis.

8. The method of claim 1, wherein the cancer to be treated is ovarian cancer, pancreatic cancer, or breast cancer.

9. The method of claim 1, wherein the cancer to be treated is a solid tumor type of cancer, a non-solid tumor type of cancer, a hematopoietic cancer, or a leukemia.

10. The method of claim 1, wherein the cancer to be treated is selected from the group consisting of leukemias, carcinomas, sarcomas, lymphomas, cancers that begin in the skin, cancers that begin in tissues that line or cover internal organs, thyroid cancer, neck cancer, skin cancer, melanoma, kidney cancer, gastrointestinal cancers, cancer of the digestive system, esophageal cancer, gallbladder cancer, liver cancer, pancreatic cancer, stomach cancer, small intestine cancer, large intestine (colon) cancer, rectal cancer, gynecological cancers, cervical cancer, ovarian cancer, uterine cancer, vaginal cancer, vulvar cancer, prostate cancer, bladder cancer, endometrial cancer, breast cancer, and lung cancer.

11. The method of claim 1, wherein the composition is administered topically, orally, intranasaly, subcutaneously, intradermally, intravenously, intraperitoneally, intramuscularly, epidurally, parenterally, intranasally, and/or intracranially.

12. The method of claim 1, wherein the subject is human.

13. A method for treating a brain cancer comprising administering to a subject in need a combination of:
(a) a composition comprising at least one compound selected from the group consisting of:
(i) Hematopoietic Signal peptide-containing Secreted 1 (HSS1);
(ii) Hematopoietic Signal peptide-containing Membrane domain-containing 1 (HSM1);
(iii) a peptide having at least about 80% homology to HSS1, wherein the peptide exhibits anti-angiogenic activity;
(iv) a peptide having at least about 80% homology to HSM1, wherein the peptide exhibits anti-angiogenic activity;
(v) a HSS1 fragment comprising at least 4 contiguous amino acids from the HSS1 amino acid sequence, wherein the fragment exhibits anti-angiogenic activity;
(vi) a HSM1 fragment comprising at least 4 contiguous amino acids from the HSM1 amino acid sequence, wherein the fragment exhibits anti-angiogenic activity; and
(vii) any combination thereof; and
(b) a conventional cancer therapy which is surgery, chemotherapy, radiation therapy, or any combination thereof.

14. The method of claim 13, wherein the brain cancer is selected from the group consisting of glioma, neuroblastoma, astrocytoma, oligodendroglioma, ependymoma, meningiomas, acoustic neuroma/schwannomas, glioblastoma multiforme, and medulloblastoma

15. The method of claim 13, wherein the brain cancer is a primary brain cancer.

16. The method of claim 13, wherein the brain cancer is a secondary brain cancer which has metastatized from a non-brain cancer.

17. The method of claim 13, wherein the composition is administered before, during, or after the conventional cancer therapy.

18. The method of claim 13, wherein the conventional cancer therapy is radiation therapy, and the composition radiosensitizes the cancer.

19. The method of claim 13, wherein the cancer patient population to be treated is a carrier of BRCA1-2.

20. The method of claim 13, wherein the composition increases the survival of the subject.

21. The method of claim 13, wherein the subject has a tumor, and the composition results in slowing or inhibition of tumor growth, progression, and/or metastasis.

22. The method of claim 13, wherein the composition is administered topically, orally, intranasaly, subcutaneously, intradermally, intravenously, intraperitoneally, intramuscularly, epidurally, parenterally, intranasally, and/or intracranially.

23. The method of claim 13, wherein the subject is human.

24. A method for treating inflammatory diseases by reducing inflammatory cell invasion by anti-angiogenic activity comprising administering to a subject in need a composition comprising at least one compound selected from the group consisting of:
(a) Hematopoietic Signal peptide-containing Secreted 1 (HSS1);
(b) Hematopoietic Signal peptide-containing Membrane domain-containing 1 (HSM1);
(c) a peptide having at least about 80% homology to HSS1, wherein the peptide exhibits anti-angiogenic activity;
(d) a peptide having at least about 80% homology to HSM1, wherein the peptide exhibits anti-angiogenic activity;
(e) a HSS1 fragment comprising at least 4 contiguous amino acids from the HSS1 amino acid sequence, wherein the fragment exhibits anti-angiogenic activity;
(f) a HSM1 fragment comprising at least 4 contiguous amino acids from the HSM1 amino acid sequence, wherein the fragment exhibits anti-angiogenic activity; and
(g) any combination thereof.

25. The method of claim 24, wherein the inflammatory disease is Arthritis, Crohn's disease, Psoriasis, or Endometriosis.
